(19) United States
(12) Patent Application Publication
(10) Pub. No.: US 2023/0309800 A1
(43) Pub. Date: Oct. 5, 2023

(54) SYSTEM AND METHOD OF SCANNING TEETH FOR RESTORATIVE DENTISTRY

(71) Applicant: Align Technology, Inc., San Jose, CA (US)

(72) Inventors: Shai Farkash, Hod Hasharon (IL); Moti Ben-Dov, Tel Mond (IL); Ran Katz, Hod Hasharon (IL); Pavel Agniashvili, Moscow (RU); Sergey Turko, Moscow (RU)

(21) Appl. No.: 18/128,883

(22) Filed: Mar. 30, 2023

Related U.S. Application Data
(60) Provisional application No. 63/326,807, filed on Apr. 1, 2022.

(51) Int. Cl. A61B 1/00; A61B 5/00

(52) U.S. Cl. CPC A61B 1/00172; A61B 1/00194; A61B 5/0088

(57) ABSTRACT

A method of intraoral scanning includes receiving a plurality of intraoral scans of a dental site during an intraoral scanning session, generating a three-dimensional (3D) surface of the dental site from the plurality of intraoral scans, identifying hard tissue and soft tissue in at least one of a) the plurality of intraoral scans of the dental site or b) the 3D surface of the dental site, and displaying a view of the 3D surface, wherein a first visualization is used to display first portions of the 3D surface identified as hard tissue and a second visualization is used to display second portions of the 3D surface identified as soft tissue.

### RELATED APPLICATIONS

The present application claims the benefit under 35 U.S.C. § 119(e) of U.S. Provisional Patent Application No. 63/326,807, filed Apr. 1, 2022, which is incorporated by reference herein. The present application is related to U.S. application Ser. No. 17/011,930, filed Sep. 30, 2020, entitled “Automated Detection, Generation And/or Correction of Dental Features in Digital Models,” which is incorporated by reference herein.

### TECHNICAL FIELD

Embodiments of the present disclosure relate to the field of dentistry and, in particular, to techniques for processing intraoral scans of preparation teeth and visualizing three-dimensional (3D) surfaces of preparation teeth and other dental sites generated from intraoral scans.

### BACKGROUND

For restorative dental work such as crowns and bridges, one or more intraoral scans may be generated of a preparation tooth and/or surrounding teeth on a patient's dental arch using an intraoral scanner. In cases of sub-gingival preparations, the gingiva covers at least portions of the margin line (also referred to herein as a finish line) and is retracted in order to fully expose the margin line. Thus, intraoral scans are generally created after a doctor packs a dental retraction cord (also referred to as packing cord or retraction cord) under the gums around the preparation tooth and then withdraws the retraction cord, briefly exposing a sub-gingival margin line. The process of packing the retraction cord between the preparation and the gums is lengthy, and can take about 10 minutes per preparation to complete. Additionally, this process is painful to the patient and can damage the gum. The intraoral scans taken after the retraction cord has been packed around the preparation tooth and then withdrawn must be taken within a narrow time window during which the gingiva collapses back over the margin line. If insufficient intraoral scans are generated before the gingiva collapses, then the process needs to be repeated. Once sufficient intraoral scans are generated, these are then used to generate a virtual three-dimensional (3D) model of a dental site including the preparation tooth and the surrounding teeth and gingiva. For example, a virtual 3D model of a patient's dental arch may be generated. The virtual 3D model may then be sent to a lab.

### SUMMARY

Multiple example implementations of the disclosure are described herein.

In a first implementation, a method comprises receiving a plurality of intraoral scans of a dental site during an intraoral scanning session, generating a three-dimensional (3D) surface of the dental site from the plurality of intraoral scans, identifying hard tissue and soft tissue in at least one of a) the plurality of intraoral scans of the dental site or b) the 3D surface of the dental site and displaying a view of the 3D surface, wherein at least one of a first visualization or a first transparency level is used for first portions of the 3D surface identified as hard tissue and at least one of a second visualization or a second transparency level is used for second portions of the 3D surface identified as soft tissue.

A second implementation may further extend the first implementation. In the second implementation, identifying the hard tissue and the soft tissue comprises processing at least one of a) the plurality of intraoral scans or b) data from the 3D surface using a trained machine learning model that has been trained to identify hard tissue and soft tissue, wherein the trained machine learning model outputs, for each location in the plurality of intraoral scans or the 3D surface, a first classification indicating hard tissue or a second classification indicating soft tissue.

A third implementation may further extend the second implementation. In the third implementation, the trained machine learning model or a second trained machine learning model further outputs, for each location, a third classification identifying the location as part of a margin line or a fourth classification identifying the location as not being part of the margin line.

A fourth implementation may extend any of the first through third implementations. In the fourth implementation, the first visualization comprises an opaque visualization and the second visualization comprises a semi-transparent visualization.

A fifth implementation may extend the fourth implementation. In the fifth implementation, the hard tissue comprises teeth and the soft tissue comprises gingiva, and wherein scanned portions of the teeth that are below a gum line are visible through the semi-transparent visualization used for the gingiva.

A sixth implementation may extend any of the first through fifth implementations. In the sixth implementation, the dental site comprises a preparation tooth, and the method further comprises identifying a margin line around at least a portion of the preparation tooth in at least one of a) one or more of the plurality of intraoral scans or b) data from the 3D surface and displaying the margin line on the 3D surface using one or more additional visualizations.

A seventh implementation may extend the sixth implementation. In the seventh implementation, the method further comprises grading a plurality of portions of the margin line, wherein the one or more additional visualizations comprises a plurality of visualizations, wherein each of the plurality of visualizations is associated with a different margin line grade, and wherein each portion of the plurality of portions of the margin line are displayed using a respective one of the plurality of visualizations that is associated with the margin line grade for that portion.

An eighth implementation may extend the seventh implementation. In the eighth implementation, the margin line is identified by processing at least one of a) the plurality of intraoral scans or b) data from the 3D surface using a trained machine learning model that has been trained to identify the margin line, wherein for each location the trained machine learning model outputs an indication of whether the location depicts a margin line and a confidence rating, wherein locations identified as depicting the margin line and having a confidence rating that meets or exceeds a confidence threshold are assigned a higher margin line grade than locations identified as depicting the margin line and having a confidence rating that does not meet the confidence threshold.

A ninth implementation may extend the eighth implementation. In the ninth implementation, the method further comprises: determining, for each of the locations, whether the location is associated with at least one of: a) a curvature sharpness that is below a curvature sharpness threshold, b) an s-shaped curve, c) a void in the margin line, or d) fewer than a threshold number of intraoral scans; and reducing grades for those locations that are associated with at least one of a) a curvature sharpness that is below a curvature sharpness threshold, b) an s-shaped curve, c) a void in the margin line, or d) fewer than the threshold number of intraoral scans.

A tenth implementation may extend any of the first through ninth implementations. In the tenth implementation, one or more intraoral scans of the plurality of intraoral scans include a representation of a dental tool, the method further comprising: identifying the dental tool in at least one of a) the one or more intraoral scans that include the representation of the dental tool or b) the 3D surface; and removing the dental tool from at least one of a) the one or more intraoral scans or b) the 3D surface.

An 11^{th} implementation may extend any of the first through 10^{th} implementations. In the 11^{th} implementation, the method further comprises: receiving an additional intraoral scan of the dental site; adding data from the additional intraoral scan to the 3D surface; updating the view of the 3D surface, wherein the data from the additional intraoral scan is semi-transparent in the updated view of the 3D surface; subsequently segmenting the data from the additional intraoral scan into hard tissue and soft tissue; and subsequently updating the view of the 3D surface such that the data from the additional intraoral scan associated with hard tissue is opaque and the data from the additional intraoral scan associated with soft tissue remains semi-transparent.

A 12^{th} implementation may extend any of the first through 11^{th} implementations. In the 12^{th} implementation, the method further comprises: receiving an additional intraoral scan of the dental site; adding data from the additional intraoral scan to the 3D surface; updating the view of the 3D surface, wherein the data from the additional intraoral scan is semi-transparent in the updated view of the 3D surface; subsequently identifying at least one of moving tissue or a dental tool in the data from the additional intraoral scan; removing at least one of the moving tissue or the dental tool from the 3D surface; and subsequently updating the view of the 3D surface to reflect at least one of the removed moving tissue or the removed dental tool.

A 13^{th} implementation may extend the 12^{th} implementation. In the 13^{th} implementation, the method further comprises locking one or more regions of at least one of a) the plurality of intraoral scans or b) the 3D surface that are identified as hard tissue, wherein those regions that are identified as the moving tissue or the dental tool and that are locked are not removed from the 3D surface.

A 14^{th} implementation may extend any of the first through 13^{th} implementations. In the 14^{th} implementation, the method further comprises: determining whether or not the dental site comprises a preparation tooth; using a first moving tissue detection algorithm to identify and remove moving tissue from at least one of the plurality of intraoral scans or the 3D surface responsive to determining that a preparation tooth is not detected; and using a second moving tissue detection algorithm to identify and remove moving tissue from at least one of the plurality of intraoral scans or the 3D surface responsive to determining that a preparation tooth is detected, wherein the second moving tissue detection algorithm is more aggressive at identifying moving tissue than the first moving tissue detection algorithm.

A 15^{th} implementation may extend the 14^{th} implementation. In the 15^{th} implementation, the method further comprises receiving user input indicating that the dental site comprises a preparation tooth.

A 16^{th} implementation may extend the 14^{th} or 15^{th} implementation. In the 16^{th} implementation, the method further comprises analyzing at least one of the plurality of intraoral scans or the 3D surface to determine that the dental site comprises a preparation tooth.

A 17^{th} implementation may extend any of the 1^{st} through the 16^{th} implementations. In the 17^{th} implementation, the first transparency level comprises 0-50% transparency, and wherein the second transparency level comprises 51-100% transparency.

An 18^{th} implementation may extend any of the 1^{st} through the 17^{th} implementations. In the 18^{th} implementation, the second transparency level comprises 100% transparency, and the second portions of the 3D surface identified as soft tissue are not visible due to the 100% transparency.

An 19^{th} implementation may extend any of the 1^{st} through the 18^{th} implementations. In the 19^{th} implementation, the 3D surface is a 3D surface of a preparation tooth, the method further comprising overlaying the 3D surface onto a second 3D surface of the dental arch that includes the preparation tooth, wherein gums from the second 3D surface are shown using a semi-transparent visualization.

A 20^{th} implementation may extend any of the 1^{st} through the 19^{th} implementations. In the 20^{th} implementation, the method further comprises: receiving a first user input specifying at least one of the first visualization or the first level of transparency; and receiving a second user input specifying at least one of the second visualization or the second level of transparency.

A 21^{st} implementation may extend any of the 1^{st} through the 20^{th} implementations. In the 21^{st} implementation, the method further comprises: receiving a user input of a coordinate; determining a first tooth closest to the coordinate; using at least one of the first visualization or the first transparency level for displaying the first tooth closest to the coordinate; and using at least one of the second visualization or the second transparency level for displaying a second tooth.

A 22^{nd} implementation may extend the 21^{st} implementation. In the 22^{nd} implementation, the method further comprises: receiving a new user input of a new coordinate; determining that the second tooth is a closest tooth to the new coordinate; using at least one of the first visualization or the first transparency level for displaying the second tooth closest to the new coordinate; and using at least one of the second visualization or the second transparency level for displaying the first tooth.

A 23^{rd} implementation may extend the 21^{st} or 22^{nd} implementation. In the 23^{rd} implementation, at least one of a mesial surface or a distal surface of the first tooth is visible through the second tooth displayed using at least one of the second visualization or the second transparency level.

A 24^{th} implementation may extend any of the 21^{st} through 23^{rd} implementations. In the 24^{th} implementation, receiving the user input of the coordinate comprises receiving user input dragging a hint feature to the coordinate.

A 25^{th} implementation may extend any of the 21^{st} through 24^{rd} implementations. In the 25^{th} implementation, the first tooth is a preparation tooth having a margin line, and wherein determining the first tooth closest to the coordinate comprises: identifying a margin line of the preparation tooth; determining that a point on the 3D surface closet to the coordinate is within the margin line in a plane; and classifying points on the preparation tooth that are within the margin line as the preparation tooth.

A 26^{th} implementation may extend any of the 21^{st} through 24^{th} implementations. In the 25^{th} implementation, determining the first tooth closest to the coordinate comprises: performing one or more morphological operations to divide the 3D surface into a plurality of parts that correspond to distinct teeth; finding a point on the 3D surface closest to the coordinate; and selecting a part associated with the first tooth that comprises the point on the 3D surface closest to the coordinate.

In a 27^{th} implementation, a computing device comprising a memory and a processing device performs the operations of any of the 1^{st} through 26^{th} implementations.

In a 28^{th} implementation, a non-transitory computer readable medium comprises instructions that, when executed by a processing device, causes the processing device to execute the method of any of the 1^{st} through 26^{th} implementations.

In a 29^{th} implementation, a non-transitory computer readable medium comprises instructions that, when executed by a processing device, cause the processing device to perform operations comprising: displaying a view of a 3D surface of a dental site during an intraoral scanning session, the 3D surface having been generated based on one or more previously received intraoral scans captured during the intraoral scanning session, wherein a first visualization is used to display first portions of the 3D surface that represent hard tissue and a second visualization is used to display second portions of the 3D surface identified as soft tissue; receiving a new intraoral scan of the dental site during the intraoral scanning session; adding data from the new intraoral scan to the 3D surface; and updating the view of the 3D surface, wherein the data from the new intraoral scan is semi-transparent in the updated view of the 3D surface until the data from the new intraoral scan is analyzed.

A 30^{th} implementation may extend the 29^{th} implementation. In the 30^{th} implementation, the operations further comprise: analyzing the data from the new intraoral scan to identify hard tissue and soft tissue in the new intraoral scan of the dental site; and subsequently updating the view of the 3D surface such that the data from the new intraoral scan associated with hard tissue is opaque and the data from the new intraoral scan associated with soft tissue remains semi-transparent.

A 31^{st} implementation may extend the 30^{th} implementation. In the 31^{st} implementation, the analyzing is performed by processing the data from the new intraoral scan using a trained machine learning model that has been trained to identify hard tissue and soft tissue, wherein the trained machine learning model outputs, for each location in the data from the new intraoral scan, a first classification indicating hard tissue or a second classification indicating soft tissue.

A 32^{nd} implementation may extend the 30^{th} or 31^{st} implementation. In the 32^{nd} implementation, the hard tissue comprises teeth and the soft tissue comprises gingiva, and wherein scanned portions of the teeth that are below a gum line are visible through the semi-transparent gingiva.

A 33^{rd} implementation may extend any of the 29ths through 32^{nd} implementations. In the 33^{rd} implementation, the dental site comprises a preparation tooth, the operations further comprising: identifying a margin line around at least a portion of the preparation tooth in the 3D surface; and emphasizing the margin line on the 3D surface using one or more visualizations.

A 34^{th} implementation may further extend the 33^{rd} implementation. In the 34^{th} implementation, the operations further comprise: grading a plurality of portions of the margin line; wherein the one or more visualizations comprises a plurality of visualizations, wherein each of the plurality of visualizations is associated with a different margin line grade, and wherein each portion of the plurality of portions of the margin line are displayed using a respective one of the plurality of visualizations that is associated with the margin line grade for that portion.

A 35^{th} implementation may further extend the 34^{th} implementation. In the 35^{th} implementation, the margin line is identified by processing data from the 3D surface using a trained machine learning model that has been trained to identify the margin line, wherein for each location the trained machine learning model outputs an indication of whether the location depicts a margin line and a confidence rating, wherein locations identified as depicting the margin line and having a confidence rating that meets or exceeds a confidence threshold are assigned a higher margin line grade than locations identified as depicting the margin line and having a confidence rating that does not meet the confidence threshold.

A 36^{th} implementation may further extend the 35^{th} implementation. In the 36^{th} implementation, the operations further comprise: determining, for each of the locations, whether the location is associated with at least one of: a) a curvature sharpness that is below a curvature sharpness threshold, b) an s-shaped curve, c) a void in the margin line, or d) fewer than a threshold number of intraoral scans; and reducing grades for those locations that are associated with at least one of a) a curvature sharpness that is below a curvature sharpness threshold, b) an s-shaped curve, c) a void in the margin line, or d) fewer than a threshold number of intraoral scans.

A 37^{th} implementation may further extend any of the 29^{th} through 36^{th} implementations. In the 37^{th} implementation, the operations further comprise: identifying at least one of moving tissue or a dental tool in the data from the new intraoral scan; removing at least one of the moving tissue or the dental tool from the 3D surface; and subsequently updating the view of the 3D surface to reflect at least one of the removed moving tissue or the removed dental tool.

A 38^{th} implementation may further extend the 37^{th} implementation. In the 38^{th} implementation, the operations further comprise: locking one or more regions of at least one of the new intraoral scan or the 3D surface that are identified as hard tissue, wherein those regions that are identified as the moving tissue or the dental tool and that are locked are not removed from the 3D surface.

In a 39^{th} implementation, the non-transitory computer readable medium of any of the 29^{th} through 38^{th} implementations is a component of a computing device that includes the non-transitory computer readable medium and a processing device that performs the operations.

In a 40^{th} implementation, a system comprises an intraoral scanner configured to generate a plurality of intraoral scans of a dental site, and a computing device comprising a memory and a processor. The computing is configured to receive the plurality of intraoral scans of the dental site; generate a three-dimensional (3D) surface of the dental site from the plurality of intraoral scans; display a view of the 3D surface of the dental site; determine whether or not the dental site comprises a preparation tooth; use a first moving tissue detection algorithm to identify and remove moving tissue from at least one of the plurality of intraoral scans or the 3D surface responsive to determining that a preparation tooth is not detected; and use a second moving tissue detection algorithm to identify and remove moving tissue from at least one of a) the plurality of intraoral scans or b) the 3D surface responsive to determining that a preparation tooth is detected, wherein the second moving tissue detection algorithm identifies some locations as moving tissue that the first moving tissue detection algorithm identifies as not being moving tissue.

A 41^{st} implementation may extend the 40^{th} implementation. In the 41^{st} implementation, the computing device is further to receive user input indicating that the dental site comprises a preparation tooth.

A 42^{nd} implementation may extend the 40^{th} or 41^{st} implementations. In the 42^{nd} implementation, the computing device is further to analyze at least one of the plurality of intraoral scans or the 3D surface to determine that the dental site comprises a preparation tooth.

A 43^{rd} implementation may extend any of the 40^{th} through 42^{nd} implementations. In the 43^{rd} implementation, the computing device is further to analyze data from plurality of intraoral scans to identify hard tissue and soft tissue, wherein a first visualization is used to display first portions of the 3D surface that represent hard tissue and a second visualization is used to display second portions of the 3D surface identified as soft tissue.

A 44^{th} implementation may extend the 43^{rd} implementation. In the 44^{th} implementation, analyzing the data is performed by processing the data using a trained machine learning model that has been trained to identify hard tissue and soft tissue, wherein the trained machine learning model outputs, for each location in the data, a first classification indicating hard tissue or a second classification indicating soft tissue.

A 45^{th} implementation may extend the 43^{rd} or 44^{th} implementation. In the 45^{th} implementation, the first visualization comprises an opaque visualization, wherein the second visualization comprises a semi-transparent visualization, wherein the hard tissue comprises teeth, wherein the soft tissue comprises gingiva, and wherein scanned portions of the teeth that are below a gum line are visible through the semi-transparent visualization of the gingiva.

In a 46^{th} implementation, a method comprises: receiving a plurality of intraoral scans of a dental site during an intraoral scanning session; generating a three-dimensional (3D) surface of the dental site from the plurality of intraoral scans, the dental site comprising a plurality of teeth; receiving a coordinate; determining a first tooth of the plurality of teeth that is closest to the coordinate; using a first visualization for displaying the first tooth closest to the coordinate; and using a second visualization for displaying a remainder of the plurality of teeth.

A 47^{th} implementation may further extend the 46^{th} implementation. In the 47^{th} implementation, the method further comprises performing one or more morphological operations to divide the 3D surface into the plurality of teeth.

A 48^{th} implementation may further extend the 47^{th} implementation. In the 48^{th} implementation, the one or more morphological operations comprise at least one of an erode operation or a dilate operation.

A 49^{th} implementation may further extend any of the 46^{th} through 48^{th} implementations. In the 49^{th} implementation, the method further comprises: receiving a new coordinate; determining that a second tooth of the plurality of teeth is a closest tooth to the new coordinate; using the first visualization for displaying the second tooth closest to the new coordinate; and using the second visualization for displaying the first tooth.

A 50^{th} implementation may further extend any of the 46^{th} through 49^{th} implementations. In the 50^{th} implementation, at least one of a mesial surface or a distal surface of the first tooth is visible through the remainder of the plurality of teeth displayed using at least one of the second visualization.

A 51^{st} implementation may further extend any of the 46^{th} through 50^{th} implementations. In the 51^{st} implementation, the coordinate is received via user input.

A 52^{nd} implementation may further extend the 51^{st} implementation. In the 52^{nd} implementation, receiving the user input of the coordinate comprises receiving user input dragging a hint feature to the coordinate.

A 53^{rd} implementation may further extend any of the 46^{th} through 52^{nd} implementations. In the 53^{rd} implementation, the first tooth is a preparation tooth having a margin line, and wherein determining the first tooth closest to the coordinate comprises: identifying a margin line of the preparation tooth; and determining that a point on the 3D surface closest to the coordinate is within the margin line; and classifying points on the preparation tooth that are within the margin line as the preparation tooth.

A 54^{th} implementation may further extend any of the 46^{th} through 53^{rd} implementations. In the 54^{th} implementation, the first visualization comprises an opaque visualization, and wherein the second visualization comprises a semi-transparent visualization.

In a 55^{th} implementation, a computing device comprising a memory and a processing device performs the operations of any of the 46^{th} through 54^{th} implementations.

In a 56^{th} implementation, a non-transitory computer readable medium comprises instructions that, when executed by a processing device, causes the processing device to execute the method of any of the 46^{th} through 54^{th} implementations.

### BRIEF DESCRIPTION OF THE DRAWINGS

Embodiments of the present disclosure are illustrated by way of example, and not by way of limitation, in the figures of the accompanying drawings.

FIG. 1 illustrates one embodiment of a system for performing intraoral scanning and/or generating a virtual three-dimensional model of an intraoral site.

FIG. 2A illustrates a flow diagram for a method of scanning a dental site, in accordance with an embodiment.

FIG. 2B illustrates a flow diagram for a method of displaying a 3D surface of a dental site, in accordance with an embodiment.

FIGS. 3A-D illustrate views of a 3D surface of a first dental site generated from intraoral scan data during an intraoral scan session, in accordance with an embodiment.

FIGS. 3E-H illustrate views of a 3D surface of a second dental site generated from intraoral scan data during an intraoral scan session, in accordance with an embodiment.

FIGS. 4A-G illustrate a graphical user interface displaying views of a 3D surface of a dental site generated from intraoral scan data during an intraoral scan session, in accordance with an embodiment.

FIG. 5A illustrates a flow diagram for a method of scanning a preparation tooth, in accordance with an embodiment.

FIG. 5B illustrates a flow diagram for a method of using two different scanning modes for scanning different portions of an oral cavity, in accordance with an embodiment.

FIG. 6A illustrates a flow diagram for a method of resolving conflicting scan data of a dental site, in accordance with an embodiment.

FIG. 6B illustrates resolution of conflicting scan data of a dental site, in accordance with an embodiment.

FIG. 7A illustrates a flow diagram for a partial retraction method of scanning a preparation tooth, in accordance with an embodiment.

FIG. 7B illustrates another flow diagram for a partial retraction method of scanning a preparation tooth, in accordance with an embodiment.

FIGS. 7C-G illustrate a partial retraction method of scanning a preparation tooth, in accordance with an embodiment.

FIG. 8 illustrates a flow diagram for a method of identifying and grading a margin line for a preparation tooth, in accordance with an embodiment.

FIG. 9 illustrates an example workflow for generating an accurate virtual 3D model of a dental site and manufacturing a dental prosthetic from the virtual 3D model, in accordance with embodiments of the present disclosure.

FIG. 10 illustrates a flow diagram for a method of segmenting intraoral scan data into various classes and updating a 3D surface based on the classes, in accordance with an embodiment.

FIG. 11 illustrates workflows for training machine learning models and applying the trained machine learning models during intraoral scanning, in accordance with embodiments of the present disclosure.

FIG. 12 illustrates a flow diagram for a method of training a machine learning model to perform segmentation of intraoral scans and/or 3D surfaces generated from intraoral scans, in accordance with an embodiment.

FIG. 13 illustrates a block diagram of an example computing device, in accordance with embodiments of the present disclosure.

### DETAILED DESCRIPTION

Described herein are methods and systems for generating 3D surfaces and virtual 3D models of dental sites based on intraoral scan data, in accordance with embodiments of the present disclosure. Certain embodiments are directed to a user interface of an intraoral scanning system and/or intraoral scanning application. The user interface may present different portions of a 3D surface generated from intraoral scan data in different manners according to classifications of those different portions. In some embodiments, processing logic performs point-level (e.g., pixel level) or patch-level classification of a 3D surface of a dental site and/or of intraoral scans and/or projections of a 3D surface of a dental site. Points or pixels may be classified, for example, as soft tissue (e.g., such as gingiva) or as hard tissue (e.g., such as teeth). Different visualizations may then be used to display soft tissue and hard tissue. For example, an opaque visualization may be used to present hard tissue and a transparent or semi-transparent visualization may be used to present soft tissue. In one embodiment, an opaque visualization is used to present hard tissue, and soft tissue is fully transparent (e.g., such that the soft tissue is not visible in a display).

By displaying or presenting soft tissue using a transparent or semi-transparent visualization and displaying hard tissue using an opaque visualization, a user interface for an intraoral scanning system enables a dental practitioner to see both a patient's gingiva and see and understand what hard tissue lies below a gingival line. Accordingly, the dental practitioner can view a sub-gingival portion of a tooth, including a sub-gingival margin line on a 3D surface of a preparation tooth, while also viewing how the gingiva overlies the sub-gingival portions of the tooth. In embodiments in which soft tissue is hidden (e.g., 100% transparent), the dental practitioner may view the tooth (including sub-gingival portions of the tooth) without any portion of the scanned tooth being obscured by gingiva. Accordingly, the user interface of the intraoral scanning system provides a new user experience that enables the dental practitioner to more quickly and accurately generate a 3D model of a preparation tooth.

Some embodiments enable the acquisition of accurate intraoral scan data of a preparation tooth, including a margin line for a preparation tooth. For example, embodiments cover techniques for exposing just portions of the margin line at a time and generating intraoral scans of the exposed portions of the margin line without the use of a retraction cord (which exposes all of the margin line at one time). Some embodiments provide multiple scanning modes, where one scanning mode is for scanning of a preparation tooth and another scanning mode is for scanning teeth other than preparation teeth. In some embodiments, tools such as a dental probe, spatula, stream of air, etc. may be used to expose a small region of the margin line while it is scanned. This process may be repeated for all of the regions of the margin line until the entire margin line is scanned.

For many prosthodontic procedures (e.g., to create a crown, bridge, veneer, etc.), an existing tooth of a patient is ground down to a stump. The ground tooth is referred to herein as a preparation tooth, or simply a preparation. The preparation tooth has a margin line (also referred to as a margin line), which is a border between a natural (unground) portion of the preparation tooth and the prepared (ground) portion of the preparation tooth. The preparation tooth is typically created so that a crown or other prosthesis can be mounted or seated on the preparation tooth. In many instances, the margin line of the preparation tooth is sub-gingival (below the gum line). While the term preparation typically refers to the stump of a preparation tooth, including the margin line and shoulder that remains of the tooth, the term preparation herein also includes artificial stumps, pivots, cores and posts, or other devices that may be implanted in the intraoral cavity so as to receive a crown or other prosthesis. Embodiments described herein with reference to a preparation tooth also apply to other types of preparations, such as the aforementioned artificial stumps, pivots, and so on.

After the preparation tooth is created, a practitioner performs operations to ready that preparation tooth for scanning. Readying the preparation tooth for scanning may include wiping blood, saliva, etc. off of the preparation tooth and/or separating a patient's gum from the preparation tooth to expose the margin line. In some instances, a practitioner will insert a material (e.g., a retraction material such as a retraction cord) around the preparation tooth between the preparation tooth and the patient's gum. The practitioner will then remove the cord before generating a set of intraoral scans of the preparation tooth. The soft tissue of the gum will then revert back to its natural position, and in many cases collapses back over the margin line, after a brief time period. Accordingly, the practitioner uses an intraoral scanner to scan the readied preparation tooth and generate a set of intraoral images of the preparation tooth before the soft tissue reverts back to its natural position. The intraoral scanner may be used in a first scanning mode, referred to as a standard preparation or full retraction scanning mode, for this process.

In one embodiment, the intraoral scanner is used in a preparation scanning mode. Alternatively, the scanner may be used in a standard scanning mode. While the scanner is in the preparation scanning mode, a user may use a partial retraction scanning technique or a standard preparation scanning technique (in which a retraction cord is used to expose the margin line). For the partial retraction scanning technique, a practitioner (e.g., a dentist or doctor) may use a tool such as a dental probe, a dental spatula, a triple syringe, a tool to output a stream of air or water, etc. to partially expose the margin line around a preparation tooth being scanned. While a portion of the margin line is exposed, the intraoral scanner generates a scan of the region of the preparation tooth with the exposed portion of the margin line. The practitioner then uses the tool to expose another portion of the margin line, which is also imaged. This process continues until all of the margin line has been exposed and scanned. Different algorithms, settings, rules and criteria may be used for stitching images together for a standard scanning mode and for the preparation scanning mode. The partial retraction scanning technique may be a more efficient technique for scanning sub-gingival preparations than standard techniques such as those that use a retraction cord. The partial retraction scanning technique may be performed more quickly (e.g., on the order of 1-2 minutes, or even less than a minute) and with minimal patient discomfort. Additionally, the practitioner can perform the partial retraction scanning technique without needing to rush to avoid the gingiva collapsing back over the margin line.

The intraoral site at which a prosthesis is to be implanted generally should be measured accurately and studied carefully, so that the prosthesis such as a crown, denture or bridge, for example, can be properly designed and dimensioned to fit in place. A good fit enables mechanical stresses to be properly transmitted between the prosthesis and the jaw, and can prevent infection of the gums and tooth decay via the interface between the prosthesis and the intraoral site, for example. After the intraoral site has been scanned, a virtual 3D model (also referred to herein simply as a 3D model) of the dental site may be generated, and that 3D model may be used to manufacture a dental prosthetic. However, if the area of a preparation tooth containing the margin line lacks definition, it may not be possible to properly determine the margin line, and thus the margin of a restoration may not be properly designed.

Accordingly, embodiments disclosed herein provide automated systems and methods for analyzing, marking, and/or updating the margin line in a 3D surface of a preparation tooth generated from intraoral scan data. The 3D surface (or images generated from the 3D surface or scans used to generate the 3D surface) may be analyzed to identify the margin line. In some embodiments, the 3D surfaces, intraoral scans and/or images generated by projecting a 3D surface onto a 2D surface are analyzed using a trained machine learning model that has been trained to determine margin lines on preparation teeth. Segments of the margin line may be graded or scored, and the segments of the margin line may be marked or drawn on the 3D surface in accordance with their grades or scores. For example, margin line segments having grades or scores with values above an upper threshold may be shown with a first visualization (e.g., a green visualization), indicating that those margin line segments are acceptable. Margin line segments having grades or scores with values below a lower threshold may be shown with a second visualization (e.g., a red visualization), indicating that those margin line segments are unacceptable. Margin line segments having grades or scores that are above the lower threshold and below the upper threshold may be shown with a third visualization (e.g., a yellow visualization), indicating that those margin line segments might be acceptable. Margin line segments may be identified and graded during intraoral scanning and before a final 3D virtual model has been generated (e.g., before scanning is complete). The margin line segments may then be marked on a view of the 3D model during intraoral scanning to indicate to a dental practitioner performing intraoral scanning that further intraoral scans should be generated for those areas with low margin line scores/grades.

Additional embodiments are also described that automatically select which intraoral scans to use in generating a 3D model of a dental arch that depicts multiple dental sites (e.g., multiple teeth). Traditionally, a doctor selects a preparation tooth in scanning software, then scans the selected preparation tooth, selects another preparation tooth in the scanning software (if multiple preparation teeth are to be scanned), then scans the other preparation tooth, selects a particular arch to scan, and then scans the remainder of one or more other teeth on the dental arch with the preparation tooth or teeth. This notifies the scanning software which intraoral scans to use for each of the teeth on the dental arch in generation of the 3D model. However, the process of separately selecting and then scanning each preparation tooth and the dental arch can be cumbersome to doctors. In embodiments, the scanning software can automatically identify which intraoral scans to use for each tooth and/or which scanning mode settings (e.g., preparation scanning mode or standard scanning mode) to use without a doctor manually identifying scans to be associated with particular preparation teeth and/or scanning mode settings to use for particular teeth. Accordingly, doctors may scan preparation teeth and other teeth in any desired order and use any desired technique without manually identifying when preparation teeth are being scanned.

Embodiments are also described that automatically identify teeth represented in scans and/or 3D surfaces, identify gingiva in scans and/or 3D surfaces, identify excess material in intraoral scans and/or 3D surfaces, identify moving tissue and/or dental tools in intraoral scans and/or 3D surfaces, and/or identify margin line segments in intraoral scans and/or 3D surfaces.

Various embodiments are described herein. It should be understood that these various embodiments may be implemented as stand-alone solutions and/or may be combined. Accordingly, references to an embodiment, or one embodiment, may refer to the same embodiment and/or to different embodiments. Additionally, some embodiments are discussed with reference to restorative dentistry, and in particular to preparation teeth and margin lines. However, it should be understood that embodiments discussed with reference to restorative dentistry (e.g., prosthodontics) may also apply to corrective dentistry (e.g., orthodontia). Additionally, embodiments discussed with reference to preparation teeth may also apply to teeth generally, and not just preparation teeth. Furthermore, embodiments discussed with reference to margin lines may also apply to other dental features, such as cracks, chips, gum lines, caries, and so on.

Some embodiments are discussed herein with reference to intraoral scans. However, it should be understood that embodiments described with reference to intraoral scans also apply to lab scans or model/impression scans. A lab scan or model/impression scan may include one or more scans of a dental site or of a model or impression of a dental site, which may or may not include height maps, and which may or may not include color images.

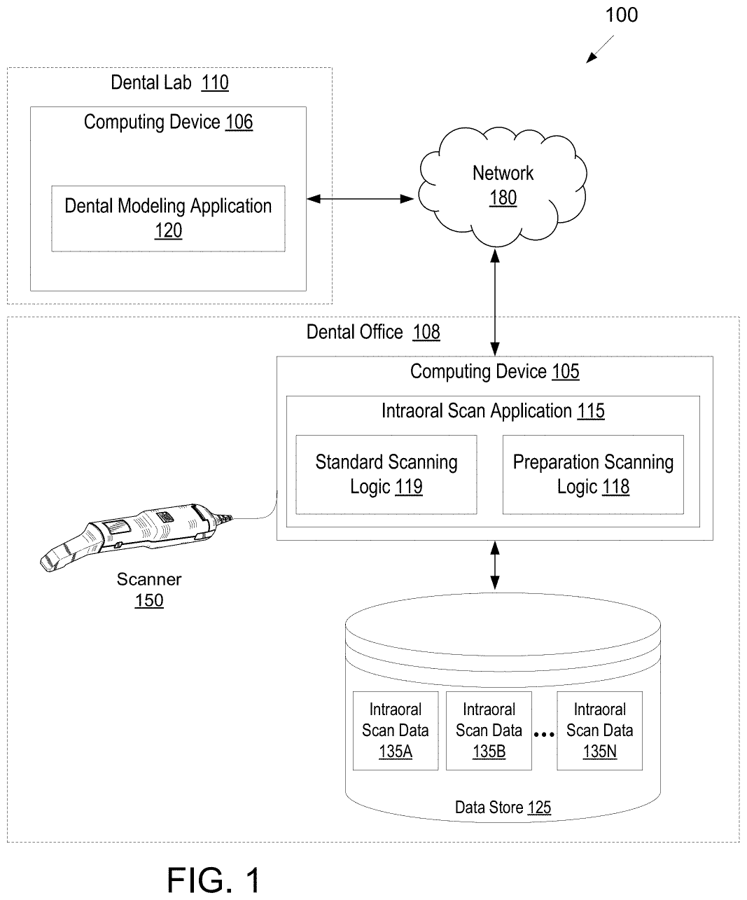

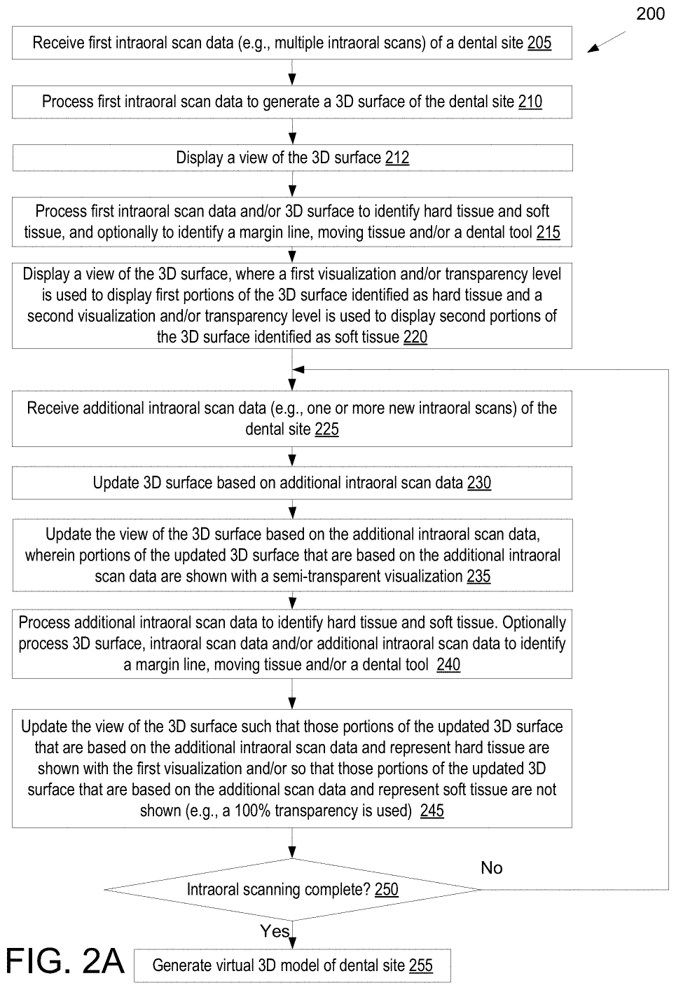

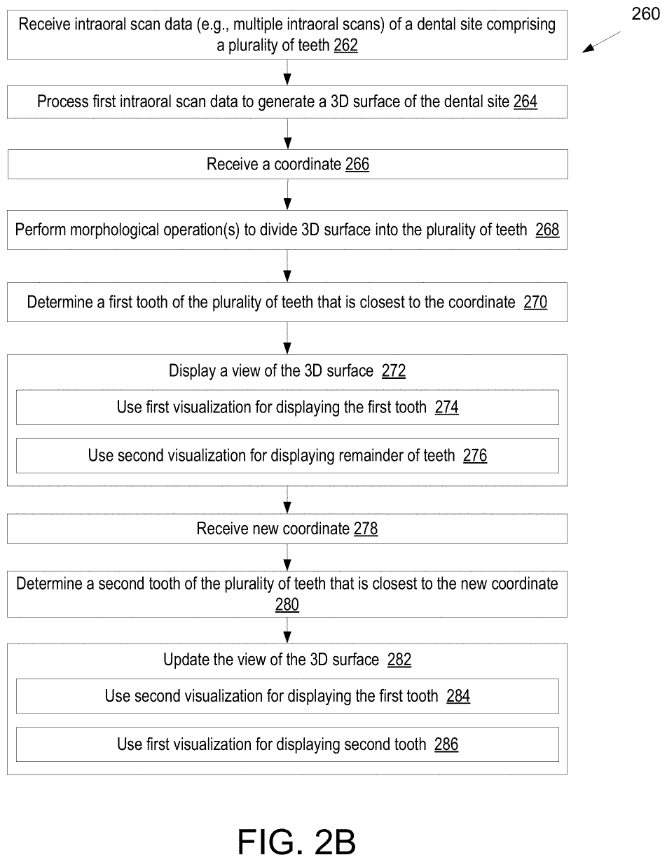

FIG. 1 illustrates one embodiment of a system 100 for performing intraoral scanning and/or generating a virtual three-dimensional (3D) model of a dental site. In one embodiment, one or more components of system 100 carries out one or more operations described below with reference to FIGS. 2-12.

System 100 may include a dental office 108 and/or a dental lab 110. The dental office 108 and the dental lab 110 each include a computing device 105, 106, where the computing devices 105, 106 may be connected to one another via a network 180. The network 180 may be a local area network (LAN), a public wide area network (WAN) (e.g., the Internet), a private WAN (e.g., an intranet), or a combination thereof.

Computing device 105 may be coupled to an intraoral scanner 150 (also referred to as a scanner) and/or a data store 125. Computing device 106 may also be connected to a data store (not shown). The data stores may be local data stores and/or remote data stores. Computing device 105 and computing device 106 may each include one or more processing devices, memory, secondary storage, one or more input devices (e.g., such as a keyboard, mouse, tablet, and so on), one or more output devices (e.g., a display, a printer, etc.), and/or other hardware components.

Intraoral scanner 150 may include a probe (e.g., a hand held probe) for optically capturing three-dimensional structures by generating intraoral scans. The intraoral scanner 150 may be used to perform intraoral scanning of a patient's oral cavity. An intraoral scan application 115 running on computing device 105 may communicate with the scanner 150 to effectuate the intraoral scanning procedure. A result of the intraoral scanning may be intraoral scan data 135A, 135B through 135N that may include one or more sets of intraoral scans and/or intraoral images. Each intraoral scan may be a two-dimensional (2D) or 3D point cloud or image that includes x, y and z information. Some intraoral scans, such as those generated by confocal scanners, include 2D height maps. In one embodiment, the intraoral scanner 150 generates numerous discrete (i.e., individual) intraoral scans. Sets of discrete intraoral scans may be merged into a smaller set of blended intraoral scans, where each blended intraoral scan is a combination of multiple discrete intraoral scans. The scanner 150 may transmit the intraoral scan data 135A, 135B through 135N to the computing device 105. Computing device 105 may store the intraoral scan data 135A-135N in data store 125.

Intraoral scan data 135A-N may optionally include one or more color images (e.g., color 2D images) and/or images generated under particular lighting conditions (e.g., ultraviolet (UV) images, infrared (IR) images and/or near-IR images).

According to an example, a user (e.g., a practitioner) may subject a patient to intraoral scanning. In doing so, the user may apply scanner 150 to one or more patient intraoral locations. The scanning may be divided into one or more segments. As an example, the segments may include an upper dental arch segment, a lower dental arch segment, a bite segment, and optionally one or more preparation tooth segments. As another example, the segments may include a lower buccal region of the patient, a lower lingual region of the patient, an upper buccal region of the patient, an upper lingual region of the patient, one or more preparation teeth of the patient (e.g., teeth of the patient to which a dental device such as a crown or other dental prosthetic will be applied), one or more teeth which are contacts of preparation teeth (e.g., teeth not themselves subject to a dental device but which are located next to one or more such teeth or which interface with one or more such teeth upon mouth closure), and/or patient bite (e.g., scanning performed with closure of the patient's mouth with the scan being directed towards an interface area of the patient's upper and lower teeth). Via such scanner application, the scanner 150 may provide intraoral scan data 135A-N to computing device 105. The intraoral scan data 135A-N may be provided in the form of intraoral scan data sets, each of which may include 3D point clouds, 2D images and/or 3D images of particular teeth and/or regions of an intraoral site. In one embodiment, separate data sets are created for the maxillary arch, for the mandibular arch, for a patient bite, and for each preparation tooth. Alternatively, a single large data set is generated (e.g., for a mandibular and/or maxillary arch). Such scans may be provided from the scanner 150 to the computing device 105 in the form of one or more points (e.g., one or more point clouds).

The manner in which the oral cavity of a patient is to be scanned may depend on the procedure to be applied thereto. For example, if an upper or lower denture is to be created, then a full scan of the mandibular or maxillary edentulous arches may be performed. In contrast, if a bridge is to be created, then just a portion of a total arch may be scanned which includes an edentulous region, the neighboring preparation teeth (e.g., abutment teeth) and the opposing arch and dentition. Additionally, the manner in which the oral cavity is to be scanned may depend on a doctor's scanning preferences and/or patient conditions.

By way of non-limiting example, dental procedures may be broadly divided into prosthodontic (restorative) and orthodontic procedures, and then further subdivided into specific forms of these procedures. Additionally, dental procedures may include identification and treatment of gum disease, sleep apnea, and intraoral conditions. The term prosthodontic procedure refers, inter alia, to any procedure involving the oral cavity and directed to the design, manufacture or installation of a dental prosthesis at a dental site within the oral cavity (intraoral site), or a real or virtual model thereof, or directed to the design and preparation of the intraoral site to receive such a prosthesis. A prosthesis may include any restoration such as crowns, veneers, inlays, onlays, implants and bridges, for example, and any other artificial partial or complete denture. The term orthodontic procedure refers, inter alia, to any procedure involving the oral cavity and directed to the design, manufacture or installation of orthodontic elements at a intraoral site within the oral cavity, or a real or virtual model thereof, or directed to the design and preparation of the intraoral site to receive such orthodontic elements. These elements may be appliances including but not limited to brackets and wires, retainers, clear aligners, or functional appliances.

For many prosthodontic procedures (e.g., to create a crown, bridge, veneer, etc.), a preparation tooth is created (e.g., by grinding a portion of a tooth to a stump). The preparation tooth has a margin line (also referred to as finish line) that can be important to proper fit of a dental prosthesis. After the preparation tooth is created, a practitioner may perform operations to ready that preparation tooth for scanning. Readying the preparation tooth for scanning may include wiping blood, saliva, etc. off of the preparation tooth and/or separating a patient's gum from the preparation tooth to expose the margin line.

In some instances, a practitioner will perform a standard preparation (full retraction) technique to expose an entirety of the margin line at once by inserting a cord around the preparation tooth between the preparation tooth and the patient's gum and then removing the cord before generating a set of intraoral scans of the preparation tooth. The soft tissue of the gum will then revert back to its natural position, and in many cases collapses back over the margin line, after a brief time period. Accordingly, some of intraoral scan data 135A-N may include images that were taken before the gum has collapsed over the margin line, and other intraoral scan data 135A-N may include images that were taken after the gum has collapsed over the martin line.

In some instances a dental practitioner performs a partial retraction scanning technique. For the partial retraction scanning technique, the gingiva is pushed aside by a tool to expose a small section of the margin line of the sub-gingival preparation. That small section is scanned, and the tool is moved, allowing the small section of the gingiva to collapse back over margin line and exposing another small section of the margin line. Accordingly, readying the preparation tooth for scanning may include using a tool to expose just a portion of the margin line, which is then scanned while it is exposed. Readying the preparation tooth may then include using the tool to expose another portion of the margin line, which is scanned while it is exposed. This process may continue until all of the margin line has been scanned.

Examples of tools that may be used to expose a portion of the margin line at a time include a dental probe, a dental spatula, a triple syringe, an air gun, dental floss, a water gun, and so on. In some embodiments, specific tools are developed for exposing one or more portions of the margin line around one or more teeth (e.g., a first tool for exposing an interproximal portion of a margin line, a second tool for exposing a lingual portion of a margin line, and so on). Different tools developed for exposing different portions of the margin line of a tooth may have protrusions, lengths, probes, spatulas, etc. with different lengths, widths, angles, and so on.

During intraoral scanning, intraoral scan application 115 may register and stitch together two or more intraoral scans (e.g., intraoral scan data 135A and intraoral scan data 135B) generated thus far from the intraoral scan session. In one embodiment, performing registration includes capturing 3D data of various points of a surface in multiple scans, and registering the scans by computing transformations between the scans. One or more 3D surfaces may be generated based on the registered and stitched together intraoral scans during the intraoral scanning. The one or more 3D surfaces may be output to a display so that a doctor or technician can view their scan progress thus far.

As each new intraoral scan is captured and registered to previous intraoral scans and/or a 3D surface, the one or more 3D surfaces may be updated, and the updated 3D surface(s) may be output to the display. In embodiments, segmentation is performed on the intraoral scans and/or the 3D surface to segment points and/or patches on the intraoral scans and/or 3D surface into one or more classifications. In one embodiment, intraoral scan application 115 classifies points as hard tissue or as soft tissue. The 3D surface may then be displayed using the classification information. For example, hard tissue may be displayed using a first visualization (e.g., an opaque visualization) and soft tissue may be displayed using a second visualization (e.g., a transparent or semi-transparent visualization).

In embodiments, separate 3D surfaces are generated for the upper jaw and the lower jaw. This process may be performed in real time or near-real time to provide an updated view of the captured 3D surfaces during the intraoral scanning process.

When a scan session or a portion of a scan session associated with a particular scanning role or segment (e.g., upper jaw role, lower jaw role, bite role, etc.) is complete (e.g., all scans for an intraoral site or dental site have been captured), intraoral scan application 115 may automatically generate a virtual 3D model of one or more scanned dental sites (e.g., of an upper jaw and a lower jaw). The final 3D model may be a set of 3D points and their connections with each other (i.e., a mesh). To generate the virtual 3D model, intraoral scan application 115 may register and stitch together the intraoral scans generated from the intraoral scan session that are associated with a particular scanning role or segment. The registration performed at this stage may be more accurate than the registration performed during the capturing of the intraoral scans, and may take more time to complete than the registration performed during the capturing of the intraoral scans. In one embodiment, performing scan registration includes capturing 3D data of various points of a surface in multiple scans, and registering the scans by computing transformations between the scans. The 3D data may be projected into a 3D space of a 3D model to form a portion of the 3D model. The intraoral scans may be integrated into a common reference frame by applying appropriate transformations to points of each registered scan and projecting each scan into the 3D space.

In one embodiment, registration is performed for adjacent or overlapping intraoral scans (e.g., each successive frame of an intraoral video). In one embodiment, registration is performed using blended scans. Registration algorithms are carried out to register two adjacent or overlapping intraoral scans (e.g., two adjacent blended intraoral scans) and/or to register an intraoral scan with a 3D model, which essentially involves determination of the transformations which align one scan with the other scan and/or with the 3D model. Registration may involve identifying multiple points in each scan (e.g., point clouds) of a scan pair (or of a scan and the 3D model), surface fitting to the points, and using local searches around points to match points of the two scans (or of the scan and the 3D model). For example, intraoral scan application 115 may match points of one scan with the closest points interpolated on the surface of another scan, and iteratively minimize the distance between matched points. Other registration techniques may also be used.

Intraoral scan application 115 may repeat registration for all intraoral scans of a sequence of intraoral scans to obtain transformations for each intraoral scan, to register each intraoral scan with previous intraoral scan(s) and/or with a common reference frame (e.g., with the 3D model). Intraoral scan application 115 may integrate intraoral scans into a single virtual 3D model by applying the appropriate determined transformations to each of the intraoral scans. Each transformation may include rotations about one to three axes and translations within one to three planes.

In many instances, data from one or more intraoral scans does not perfectly correspond to data from one or more other intraoral scans. Accordingly, in embodiments intraoral scan application 115 may process intraoral scans (e.g., which may be blended intraoral scans) to determine which intraoral scans (or which portions of intraoral scans) to use for portions of a 3D model (e.g., for portions representing a particular dental site). Intraoral scan application 115 may use data such as geometric data represented in scans and/or time stamps associated with the intraoral scans to select optimal intraoral scans to use for depicting a dental site or a portion of a dental site (e.g., for depicting a margin line of a preparation tooth). In one embodiment, images are input into a machine learning model that has been trained to select and/or grade scans of dental sites. In one embodiment, one or more scores are assigned to each scan, where each score may be associated with a particular dental site and indicate a quality of a representation of that dental site in the intraoral scans.

Additionally, or alternatively, intraoral scans may be assigned weights based on scores assigned to those scans. Assigned weights may be associated with different dental sites. In one embodiment, a weight may be assigned to each scan (e.g., to each blended scan) for a dental site (or for multiple dental sites). During model generation, conflicting data from multiple intraoral scans may be combined using a weighted average to depict a dental site. The weights that are applied may be those weights that were assigned based on quality scores for the dental site. For example, processing logic may determine that data for a particular overlapping region from a first set of intraoral scans is superior in quality to data for the particular overlapping region of a second set of intraoral scans. The first intraoral scan data set may then be weighted more heavily than the second intraoral scan data set when averaging the differences between the intraoral scan data sets. For example, the first intraoral scans assigned the higher rating may be assigned a weight of 70% and the second intraoral scans may be assigned a weight of 30%. Thus, when the data is averaged, the merged result will look more like the depiction from the first intraoral scan data set and less like the depiction from the second intraoral scan data set.

Intraoral scan application 115 may generate one or more 3D surfaces and/or 3D models from intraoral scans, and may display the 3D surfaces and/or 3D models to a user (e.g., a doctor) via a user interface. The 3D surfaces and/or 3D models can then be checked visually by the doctor. The doctor can virtually manipulate the 3D surfaces and/or 3D models via the user interface with respect to up to six degrees of freedom (i.e., translated and/or rotated with respect to one or more of three mutually orthogonal axes) using suitable user controls (hardware and/or virtual) to enable viewing of the 3D model from any desired direction. The doctor may review (e.g., visually inspect) the generated 3D surface and/or 3D model of an intraoral site and determine whether the 3D surface and/or 3D model is acceptable (e.g., whether a margin line of a preparation tooth is accurately represented in the 3D model).

In one embodiment, intraoral scan application 115 includes a standard scanning logic 119 and a preparation scanning logic 118. Standard scanning logic 119 may provide a standard scanning mode in which one or more first algorithms are used to process intraoral scan data. Preparation scanning logic 118 may provide a preparation scanning mode in which one or more second algorithms are used to process intraoral scan data. The first algorithms and second algorithms may use different rules, settings, thresholds and so on to select which images and which portions of images are used to construct portions of a virtual 3D model and/or to classify and/or segment intraoral scans and/or 3D surfaces.

Standard scanning logic 119 may provide a standard scanning mode in which one or more first algorithms are used to process intraoral scan data. The first algorithms of the standard scanning mode may be optimized to generate a virtual 3D model of a tooth other than a preparation tooth. The first algorithms may include, for example, a moving tissue detection algorithm, an excess material removal algorithm, a blending algorithm, a stitching and/or registration algorithm, and so on. Such algorithms may be configured on the assumption that the dental region being scanned is static (e.g., unmoving). Accordingly, if there is a disturbance or rapid change (e.g., a feature that is shown for only a short amount of time), the first algorithms may operate to minimize or filter out such data on the assumption that it is not part of the scanned object. For example, the first algorithms may classify such data as depictions of a tongue, cheek, finger of a doctor, tool, etc., and may not use such data in generation of a 3D model of a tooth. However, such algorithms may also remove margin line data if used on scan data showing a preparation tooth.

In one embodiment, in the standard scanning mode raw intraoral scans are received from the intraoral scanner 150, and are preliminarily registered to one another using an initial registration algorithm. A blending algorithm is then applied to the raw scans. If the scans are similar (e.g., all having a time stamp that differs by less than a time difference threshold and with surface differences that are less a surface difference threshold and/or position/orientation differences that are less than a position/orientation threshold), then the scans are blended together by the blending algorithm. For example, up to 10-20 consecutive scans taking within seconds or micro-seconds of one another may be blended together. This includes averaging the data of the scans being blended and generating a single blended scan from the averaged data. Generation of blended scans reduces the total number of scans that are processed at later scan processing stages.

The blended scans are then processed using an excess material tissue algorithm which identifies moving tissue with a size that is larger than a size threshold (e.g., over 100 or 200 microns) and then erases such excess material from the blended scans. In one embodiment, the excess material identification algorithm is a trained machine learning model (e.g., a neural network such as a convolutional neural network) that has been trained to identify such excess material. One embodiment of the machine learning model that is used for excess material removal is described in U.S. Pat. No. 11,238,586, issued Feb. 1, 2022, which is incorporated by reference herein.

Another registration algorithm then registers the blended scans together. A moving tissue algorithm then identifies moving tissue based on differences between blended scans. The moving tissue algorithm may identify moving tissue with a size that is greater than some threshold (e.g., 100 or 200 microns), and may erase such moving tissue from the blended scans. A merging algorithm may then merge together all of the remaining image data of the blended scans to generate a virtual 3D model of the preparation. The merging algorithm may average differences in data between the scans. Such differences may have a difference that is less than a threshold difference value, such as less than 0.5 mm.

Preparation scanning logic 118 may provide a preparation scanning mode in which one or more second algorithms are used to process intraoral scan data. The second algorithms of the preparation scanning mode may be optimized to generate a virtual 3D model of a preparation tooth with as clear and accurate a depiction of a margin line as possible given the scan data. The second algorithms may include, for example, a moving tissue detection algorithm, an excess material removal algorithm, a blending algorithm, a registration algorithm, a stitching or merging algorithm, and so on. In some embodiments, the second algorithms include an excess material removal algorithm, a registration algorithm and a stitching or merging algorithm. The preparation scanning mode may be optimized for use with a partial retraction scanning technique in an embodiment.

When a partial retraction scanning technique is used, the size of the exposed region of the margin line may be on the order of tens of microns. Such an exposed region of the margin line may change between scans, and may be removed by the excess material removal algorithms and/or moving tissue removal algorithms of the standard preparation scanning mode. Additionally, for partial retraction scanning there may be interrupts between scans due to the doctor moving the retraction tool between scans, whereas for standard scanning the scanning may be continuous with no interruptions. This can increase a time and difficulty in registration between scans, which may be minimized in embodiments of the second algorithms.

In some embodiments, the second algorithms include a different version of a moving tissue algorithm and/or a different version of an excess material removal algorithm than is included in the first algorithms. For example, the criteria for what constitutes excess material and/or moving tissue may be changed for the excess material removal algorithm and/or moving tissue detection algorithm.

Additionally, the first algorithms may blend scan data of multiple scans together to generate blended scans, whereas scans may not be blended using the second algorithms in some embodiments. Alternatively, the criteria for what scans can be blended together (and/or what portions of scans can be blended together) in the second algorithms may be stricter than the criteria of what scans (and/or what portions of scans) can be blended together in the first algorithms. In one embodiment, the first algorithms average the blended images at least for areas that meet some criteria (e.g., size of a matching region criterion). In one embodiment, the second algorithms omit blending for all or parts of the scans.

In one embodiment in the preparation scanning mode raw intraoral scans are received from the intraoral scanner 150. The raw scans may or may not be preliminarily registered to each other using an initial registration algorithm. In some instances blended scans are generated by blending together the raw scans. In one embodiment, blended scans are generated from multiple raw scans that were generated while a same region of a margin line was exposed. Accordingly, a blended scan may not include different scans in which different portions of a margin line are exposed. As discussed above, if a blending algorithm is used, then the blending algorithm may have stricter criteria for what data can be blended together than the blending algorithm used for the standard preparation scanning mode.

In one embodiment, the excess material identification algorithm is a trained machine learning model (e.g., a neural network such as a convolutional neural network) that has been trained to identify excess material. The machine learning model may have been specifically trained with training data that includes depictions of margin lines, so that it does not identify areas of margin lines that are changing between scans as excess material. The machine learning model may have been trained using a training dataset that includes scans of gingiva over margin lines and scans of exposed margin lines not covered by gingiva. Such a machine learning model may be trained to remove gingiva and leave exposed margin lines in an embodiment. In one embodiment, a specific excess gingiva removal algorithm (e.g., trained machine learning model) is used rather than a generic excess material removal algorithm. In one embodiment, two excess material removal algorithms are used, where one is for removing excess gingiva and the other is for removing other excess material. In one embodiment, inputs for the trained machine learning model that has been trained to remove excess gingiva are sets of scans. The trained machine learning model may determine for the sets of scans which scan data represents excess gingiva and should be removed.

One embodiment of the machine learning model that is used for excess material removal and/or excess gingiva removal is described in U.S. Pat. No. 11,238,586, except that the training dataset that is used to train the machine learning model may be different from the training dataset described in the referenced patent application. In particular, the training dataset used to train the machine learning model may include scans showing gingiva over margin lines, in which the areas with gingiva over the margin lines are labeled with point-level labeling as excess material as well as scans showing exposed margin lines with retracted gingiva that does not cover portions of the margin lines, which are labeled with point-level labels identifying areas of gingiva that are not classified as excess material. The machine learning model may be trained to perform segmentation in a manner that classifies pixels representing excess gingiva that is over margin lines as such, for example.

In one embodiment, an excess gingiva removal algorithm is used to identify and remove excess gingiva that overlies the margin line of a preparation. The excess gingiva removal algorithm may be similar to the excess material removal algorithm, but may be trained specifically to identify excess gingiva overlying a margin line for removal.

A registration algorithm registers the raw and/or blended scans (that may have been processed by the excess material removal and/or excess gingiva removal algorithms) to each other.

A merging algorithm (also referred to as a stitching algorithm) may then merge together all of the remaining image data of the scans (which may be raw or blended scans) to generate a virtual 3D model of the preparation. For any merging of multiple scans to generate a 3D model, there will inevitably be some differences between those scans that are addressed in some manner. Such differences may be determined by identifying conflicting surfaces between overlapping areas of scans, and then determining whether those conflicting surfaces meet one or more criteria. The merging algorithm may average some differences in data between the scans, and for other differences some data may be discarded. Criteria used to determine whether to average data between scans or to discard data from some of the scans include a size of a conflicting surface, differences in distances (e.g., heights or depths) between the conflicting surfaces in the scans, differences in mean or Gaussian curvature between the conflicting surfaces in the scans, and so on.

The first algorithms may include a first merging algorithm with a first size threshold and a first similarity threshold for determining whether to average together data from conflicting surfaces. The second algorithms may include a second merging algorithm with a second size threshold for determining whether to average together data from conflicting surfaces, where the second size threshold may be larger or smaller than the first size threshold. Additionally, the second merging algorithm may include a second similarity threshold that is higher than the first similarity threshold.

The size of a conflicting area may be used to determine whether to perform merging, where the size of the conflicting area must exceed some size threshold or be averaged. Thus, averaging may be performed for areas that are smaller than a threshold size and may not be performed for areas that are larger than or equal to the threshold size in embodiments for the second algorithms.

Additionally, the merging algorithm of the second algorithms may determine which image data to use for specific overlapping regions based on criteria such as distance from a scanner (depth or height) and/or curvature. For example, points from a first scan that have a larger distance from the probe and that have a greater curvature (e.g., a greater mean curvature or greater Gaussian curvature) may be selected and conflicting points from an overlapping second scan with a lower distance and/or a lower curvature may be omitted or removed. In an example, scan data may include height maps, where each height map includes a different value representing height (or conversely depth) for each pixel of the scan. Scans may be registered together, and may be determined to include overlapping pixels. The heights of these overlapping pixels may be compared, and the smaller height value (i.e., greater depth value) from one of the scans may be retained while the larger height value (i.e., smaller depth value) of the other scan may be discarded. Additionally, or alternatively, the mean curvature or Gaussian curvature may be computed for the conflicting surface from each of the scans. The scan having the higher mean curvature or higher Gaussian curvature may be selected for use in depicting that surface area, and the scan with the lower mean curvature data or lower Gaussian curvature for the conflicting surface may not be used for the surface area.

In one embodiment, a difference in the height values of the two scans is determined, and if the difference in height values for the overlapping pixels exceeds a threshold, then the smaller height value is selected. If the difference in height values of the overlapping pixels is less than the threshold, then the height values may be averaged (e.g., with a weighted or non-weighted average). Such computations may be made based on average depth values of some or all of the pixels within a conflicting surface in embodiments.

Accordingly, portions of scan data may be selected for use based on one or more criteria such as size of overlapping area (e.g., number of adjacent overlapping pixels in question), difference in height values for overlapping pixels, and/or differences in mean curvature or Gaussian curvature. For areas that are larger than a size threshold, the data for the areas from a scan that has smaller height values and/or larger mean curvature values may be selected and the data for the areas from another scan that has larger height values and/or smaller mean curvature values may be discarded or erased. This prevents data representing gingiva from being averaged with data representing a margin line, as margin lines are associated with high curvature values and gingiva are associated with much lower curvature values.

In some embodiments, data from a first set of scans is discarded, and data from a second set of scans is averaged together. For example, 7 scans may have an overlapping area with a size that meets or exceeds a size threshold. Data from 4 of the scans depicting the area may be discarded, while data from the remaining 3 scans depicting the area may be averaged. In one embodiment, a percentile is computed for the scans with an overlapping area, and those with height values within a certain percentile value (e.g., 80^{th} percentile) may be selected for removal.

Intraoral scan application 115 may include logic for automatically identifying (e.g., highlighting) a margin line in an intraoral scan and/or 3D surface of a preparation tooth. This may make it easier for the doctor to inspect the margin line for accuracy. Such marking of the margin line may be performed during intraoral scanning to enable a doctor to identify areas around a preparation tooth that would benefit from further scanning. For example, intraoral scan application 115 may mark and/or highlight specific segments of the margin line that are unclear, uncertain, and/or indeterminate. For example, segments of the margin line that are acceptable may be shown in a first color (e.g., green), while segments of the margin line that are unacceptable may be shown in a second color (e.g., red). In one embodiment, a first trained machine learning model is used to identify a margin line in a preparation tooth.

Once the doctor (e.g., dentist) has determined that a 3D model of a dental site is acceptable, the doctor may instruct computing device 105 to send the 3D model to computing device 106 of dental lab 110. Computing device 106 may include a dental modeling application 120 that may analyze the 3D model to determine if it is adequate for manufacture of a dental prosthetic. Dental modeling application 120 may include logic to identify the margin line and/or to modify the surface of one or more dental sites and/or to modify a margin line, as discussed with reference to intraoral scan application 115. If the 3D model is deemed suitable (or can be modified such that it is placed into a condition that is deemed suitable), then the dental prosthetic may be manufactured from the 3D model. If the 3D model cannot be placed into a suitable condition, then instructions may be sent back to the dental office 108 to generate one or more additional intraoral images of one or more regions of the dental site.

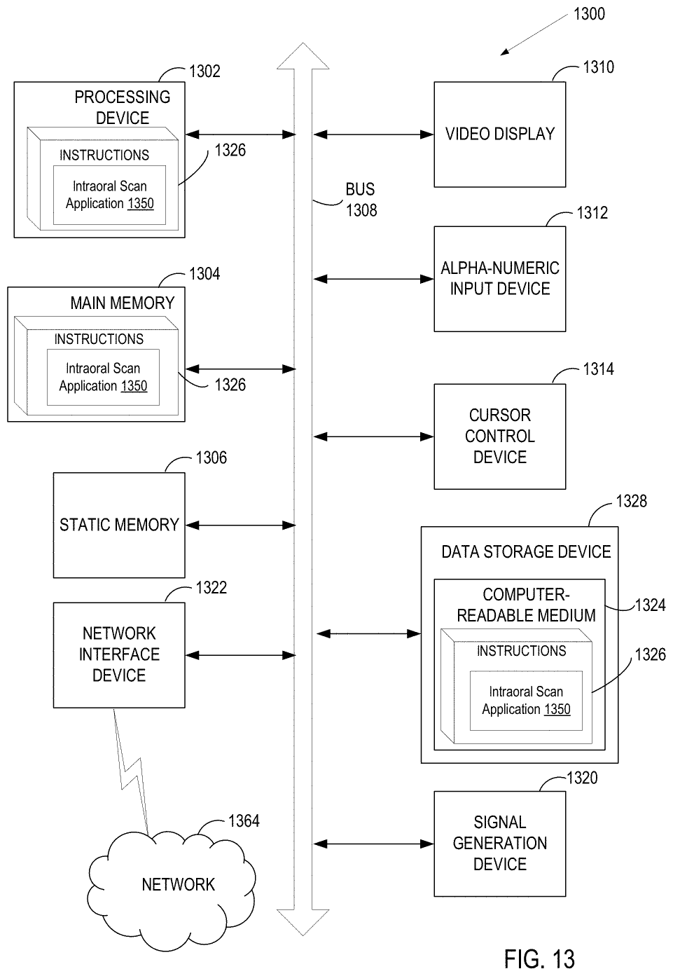

FIGS. 2A-12 illustrate methods related to intraoral scanning and generation and manipulation of virtual 3D models of dental sites. The methods may be performed by a processing logic that may comprise hardware (e.g., circuitry, dedicated logic, programmable logic, microcode, etc.), software (e.g., instructions run on a processing device to perform hardware simulation), or a combination thereof. In one embodiment, at least some operations of the methods are performed by a computing device executing an intraoral scan application, such as Intraoral Scan Application 1350 of FIG. 13. The intraoral scan application 1350 may correspond to intraoral scan application 115 of FIG. 1 in embodiments.

For simplicity of explanation, the methods are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events.

FIG. 2A illustrates a flow diagram for a method 200 of scanning a dental site, in accordance with an embodiment. At block 205 of method 200, processing logic receives first intraoral scan data (e.g., a first set of intraoral scans) of a dental site. At block 210, processing logic processes the first intraoral scan data to generate a 3D surface of the dental site. Processing the intraoral scan data may include registering and stitching intraoral scans together to generate an initial 3D surface, and subsequently registering intraoral scans to the 3D surface to expand and/or add detail to the 3D surface.

In some embodiments, at block 212 processing logic displays a view of the 3D surface before the 3D surface and/or the intraoral scans used to generate the 3D surface have been processed to identify moving tissue, to identify excess tissue, to identify dental tools, to identify hard tissue and soft tissue, and so on. The initially displayed 3D surface may be shown using a first visualization, which may be a semi-transparent visualization in embodiments. Alternatively, block 212 may be skipped, and the 3D surface may not be displayed before further processing is performed on the intraoral scan data and/or 3D surface.

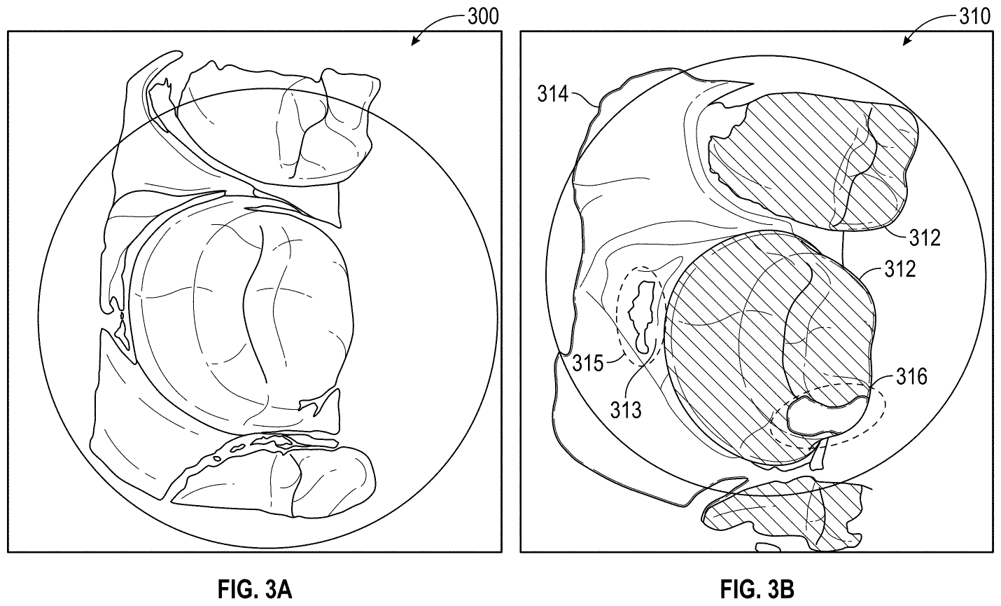

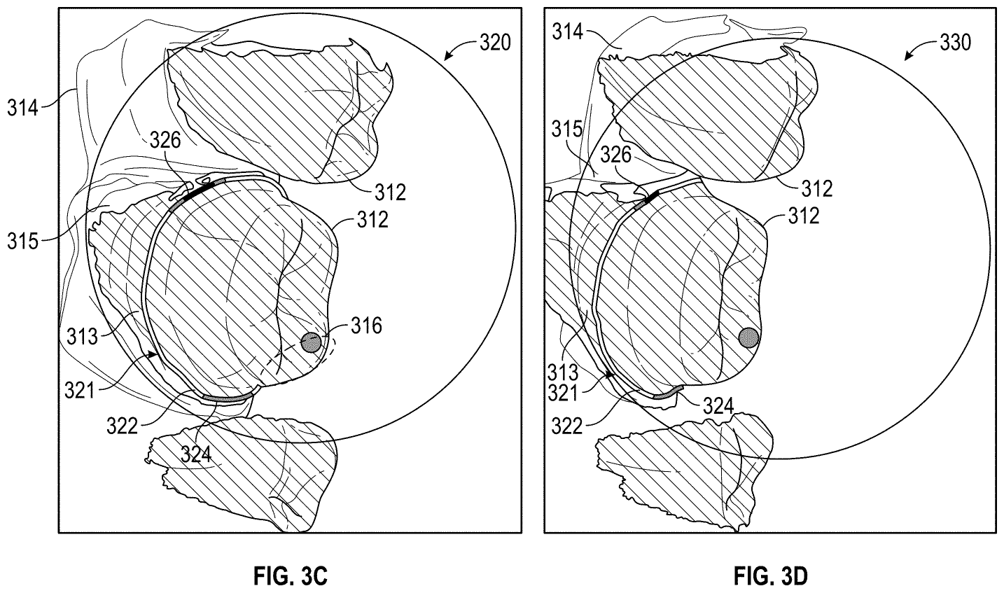

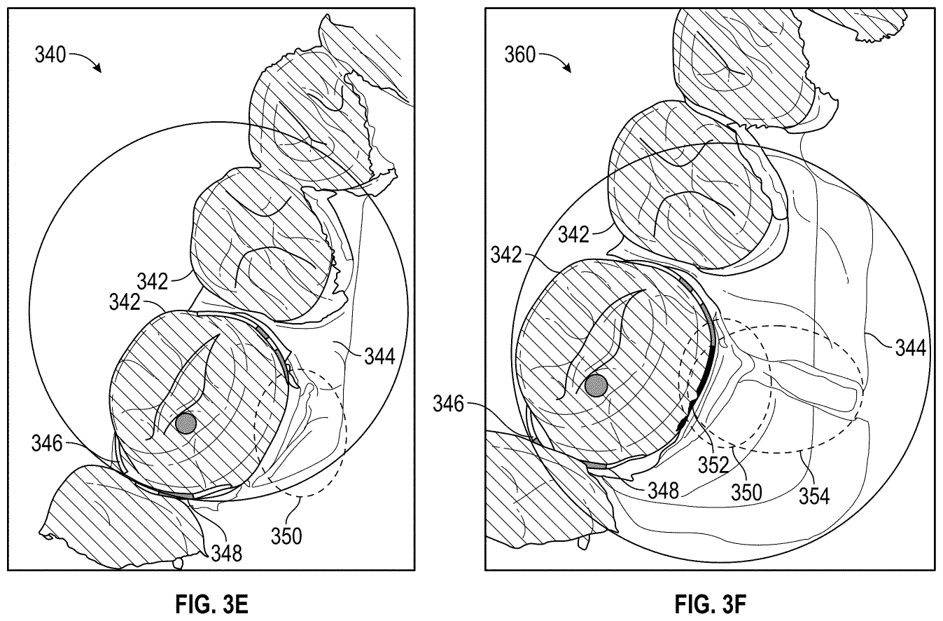

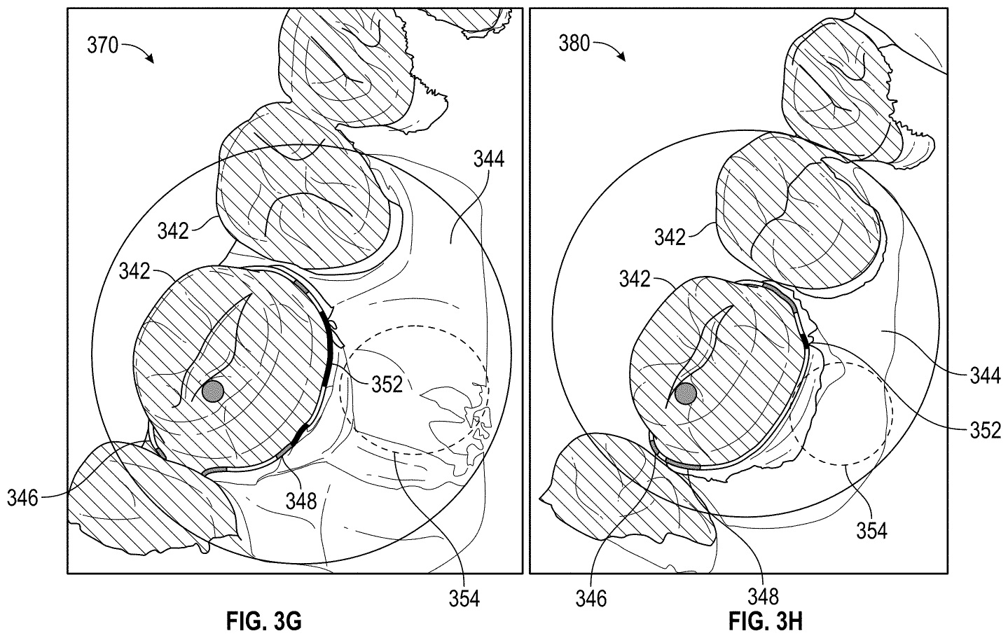

FIG. 3A illustrates an initial view of a 3D surface 300 generated from one or more intraoral scans before further processing has been performed on those intraoral scans and/or on the 3D surface to identify moving tissue, to identify excess tissue, to identify dental tools, and/or to identify hard tissue and soft tissue.

Returning to FIG. 2A, at block 215, the first intraoral scan data (e.g., the intraoral scans used to generate the 3D surface) and/or data from the 3D surface are processed to identify hard tissue and soft tissue. The data from the 3D surface may include 3D data (e.g., 3D point clouds) or 2D projections of the 3D surface. The first intraoral scan data may additionally be processed to identify points associated with a margin line and points not associated with a margin line, to identify points associated with moving tissue and/or with a dental tool, to identify points associated with excess material, and so on. Such processing may include inputting the intraoral scan data or data from the 3D surface into one or more trained machine learning models, which may output segmentation information indicating, for each point, probabilities of that point having one or more classification (e.g., a hard tissue classification, a soft tissue classification, a margin line classification, a dental tool classification, a moving tissue classification, an excess material classification, and so on). Such processing may additionally or alternatively include performing image processing and/or data processing that does not rely on trained machine learning models. In one embodiment, processing logic performs segmentation and classifies each point (e.g., each 3D point or pixel) as soft tissue or hard tissue.

At block 220, processing logic displays a view of the 3D surface (or updates a view of the 3D surface if the 3D surface was already displayed at block 212). In the view (or updated view) of the 3D surface, first portions or regions of the 3D surface that were identified as hard tissue are shown using a first visualization and/or level of transparency and second portions or regions of the 3D surface that were identified as soft tissue may shown using a second visualization and/or second level of transparency. The first visualization may include a first color (e.g., white or off-white) and the second visualization may include a second color (e.g., pink). Additionally, or alternatively, the first visualization may include a first transparency level (e.g., 0% transparent or opaque) and the second visualization may include a second transparency level (e.g., 2-1000% transparent, or semi-transparent, or fully transparent, or invisible). The second transparency level may be user adjustable in some embodiments, and may be, for example, 2%, 5%, 10%, 20%, 25%, 30%, 40%, 50%, 60%, 70%, 80%, 90%, or 100% transparent. In one embodiment, the first transparency level comprises 0-50% transparency, and the second transparency level comprises 51-100% transparency. In one embodiment, the first transparency level comprises 0% transparency and the second transparency level comprises 100% transparency, causing the portions of the 3D surface identified as soft tissue to not be visible due to the 100% transparency. In embodiments, the first visualization includes a first color and a first transparency, and the second visualization includes a second color and/or a second transparency. In some embodiments a user may select the first visualization and/or level of transparency and the second visualization and/or level of transparency. For example, the user may select whether or not they want soft tissue (e.g., gingiva) to be visible. A 100% transparency level may be set for soft tissue if a user does not wish it to be visible, and a lower transparency level (e.g., 30-50%) may be selected if the user wishes the soft tissue to be at least partially visible.

In some embodiments, the 3D surface is generated from a scan of a preparation tooth, which may include 3D surface information for adjacent teeth. A prior 3D surface may have already been generated of the dental arch that includes the preparation tooth. In such embodiments, the 3D surface of the preparation tooth may be registered to the 3D surface of the dental arch, and may be overlaid onto the 3D surface of the dental arch. The 3D surface of the dental arch may then be displayed using the second visualization and/or transparency level or a third visualization and/or transparency level. Accordingly, in some embodiments even if the gingiva of the preparation tooth from the 3D surface is not visible (due to a 100% transparency level setting), gingiva or gums from the 3D surface of the dental arch may still be visible (e.g., may be shown with a semi-transparent visualization).

FIG. 3B illustrates an updated view of a 3D surface 310 generated from one or more intraoral scans after further processing has been performed on the intraoral scans and/or on the 3D surface to identify hard tissue 312, 313 and soft tissue 314, 315. Soft tissue (e.g., gingiva or gums) 314, 315 may be shown in pink, for example. Additionally, the soft tissue 314, 315 is shown to be semi-transparent. In contrast, the hard tissue 312, 313 is shown to be opaque. Accordingly, hard tissue 313 is visible even though it is underneath the soft tissue 315. Some portions 316 of the 3D surface 310 may have been added after the 3D surface was processed to identify hard and soft tissues and/or may be based on intraoral scans that have not been processed to identify hard and soft tissue. Such portions 316 may be shown using a semi-transparent visualization in embodiments.

Returning to FIG. 2A, at block 225 processing logic receives additional intraoral scan data (e.g., one or more new intraoral scans) of the dental site. At block 230, processing logic updates the 3D surface based on the additional intraoral scan data. This may include registering and stitching the one or more new intraoral scans to the 3D surface. At block 235, processing logic updates the view of the 3D surface based on the additional intraoral scan data. Portions of the updated 3D surface that are based on the additional intraoral scan data may be shown with a semi-transparent visualization. For example, portion 316 of 3D surface 310 is shown using a semi-transparent visualization in FIG. 3B.

At block 240, processing logic processes the additional intraoral scan data to identify hard tissue and soft tissue. Processing logic may additionally or alternatively process the 3D surface (e.g., data from the 3D surface), the intraoral scan data and/or the additional intraoral scan data to identify a margin line. Processing logic may determine a confidence value for segments of the margin line, and may grade or score the segments of the margin line based on the confidence value and/or on other criteria. Processing logic may additionally or alternatively process the 3D surface, the intraoral scan data and/or the additional intraoral scan data to identify moving tissue and/or a dental tool, and/or to identify excess material.

At block 245, processing logic updates the view of the 3D surface such that those portions of the updated 3D surface that are based on the additional intraoral scan data and represent hard tissue are shown with the first visualization (e.g., as off white and/or opaque). The updated view of the 3D surface may additionally include markings for the margin line (e.g., an outline or highlight of the margin line). The markings for each segment of the margin line may be shown using a visualization associated with a grade or score assigned to that segment of the margin line. For example, a margin line segment having a high score (e.g., above an upper threshold) may be shown with a third visualization (e.g., bright green), a margin line segment having a medium score (e.g., below an upper threshold and above a lower threshold) may be shown with a fourth visualization (e.g., bright yellow), and a margin line segment having a low sore (e.g., below a lower threshold) may be shown with a fifth visualization (e.g., bright red).

FIG. 3C illustrates an updated view of an updated 3D surface 320 generated by adding information from new intraoral scans to a previously generated 3D surface after further processing has been performed on the new intraoral scans and/or on the 3D surface to identify hard tissue 312, 313 and soft tissue 314, 315. As shown, portion 316 of the 3D surface has now been classified as hard tissue, and is thus shown using the first visualization in the updated view of the updated 3D surface 320. Also shown is a marked margin line 321. The marked margin line 321 includes a first visualization 322 to show first margin line segments that are well defined, a second visualization 324 to show second margin line segments that are moderately defined, and a third visualization 326 to show third margin line segments that are poorly defined.

Returning to FIG. 2A, at block 250 processing logic may determine whether intraoral scanning is complete. Such a determination may be made based on a quality assessment of the 3D surface, based on user input, and/or based on detection of user activity (e.g., a detection that the scanner has been placed in a cradle or on a surface and is no longer in an oral cavity). If intraoral scanning is not complete, the method may return to block 225, and the process may continue by receiving and processing additional intraoral scan data.

For example, FIG. 3D shows an updated view of an updated 3D surface 330 generated by adding information from further intraoral scans to a previously generated 3D surface after further processing has been performed on the further intraoral scans and/or on the 3D surface to identify hard tissue 312, 313 and soft tissue 314, 315. As shown, the marked margin line 321 includes larger and/or more well defined segments having the first visualization 322 and fewer and/or smaller poorly defined segments having the third visualization 326 after additional intraoral scan data has been received and processed.

Returning to FIG. 2A, if at block 250 a determination is made that intraoral scanning is complete, the method continues to block 255, and a virtual 3D model may be generated for the dental site. The virtual 3D model may be similar to the 3D surface, but may be processed using algorithms that result in a higher quality surface and that use greater processing resources and take more time than those algorithms used to generate the 3D surface.

FIGS. 3E-H illustrate views of a 3D surface of a dental site generated from intraoral scan data during an intraoral scan session, in accordance with an embodiment. FIG. 3E illustrates a first view 340 of the 3D surface after a first set of intraoral scans has been processed. FIG. 3F illustrates a second view 360 of the 3D surface after a second set of intraoral scans has been processed and added to the 3D surface. FIG. 3G illustrates a third view 370 of the 3D surface after a third set of intraoral scans has been processed and added to the 3D surface. FIG. 3H illustrates a fourth view 380 of the 3D surface after a fourth set of intraoral scans has been processed and added to the 3D surface. As shown, each of the views 340, 360, 370, 380 uses a first opaque visualization 342 to represent hard tissue such as teeth and a second semi-transparent visualization 344 to represent soft tissue such as gingiva. However, in alternative embodiments soft tissue may be fully transparent, and thus may not be visible in the display. Moreover, a third visualization 346 is used to show margin line segments that are acceptable, a fourth visualization 348 is used to show margin line segments that are on the border of being acceptable, and a fifth visualization 352 is used to show margin line segments that are not acceptable. View 340 of FIG. 3E includes a first region 350 that at least partially represents moving tissue. This first region 350 is based on a recent intraoral scan, and is shown using a semi-transparent visualization. In view 360 of FIG. 3F the first region has been updated to remove tissue classified as moving tissue. Additionally, view 360 includes a second region 354 that at least partially represents additional moving tissue. This second region 354 is based on a recent intraoral scan, and is shown using a semi-transparent visualization. In view 380 of FIG. 3H the second region 354 has been further updated to remove additional tissue classified as moving tissue.

FIG. 2B illustrates a flow diagram for a method 260 of displaying a view of a 3D surface of a dental site during scanning of the dental site, in accordance with an embodiment. In embodiments, method 260 may be performed together with method 200 of FIG. 2A. At block 262 of method 260, processing logic receives intraoral scan data (e.g., a set of intraoral scans) of a dental site. At block 264, processing logic processes the intraoral scan data to generate a 3D surface of the dental site. Processing the intraoral scan data may include registering and stitching intraoral scans together to generate an initial 3D surface, and subsequently registering intraoral scans to the 3D surface to expand and/or add detail to the 3D surface.

In some embodiments, processing logic displays a view of the 3D surface before the 3D surface and/or the intraoral scans used to generate the 3D surface have been processed to identify moving tissue, to identify excess tissue, to identify dental tools, to identify hard tissue and soft tissue, and so on. The initially displayed 3D surface may be shown using a first visualization, which may be a semi-transparent visualization in embodiments. Alternatively, the 3D surface may not be displayed before further processing is performed on the intraoral scan data and/or 3D surface.

At block 266, processing logic may receive a coordinate. Processing logic may have previously received or identified (e.g., via application of machine learning) a tooth number for a preparation tooth being scanned. In embodiments, the tooth number of the preparation tooth being scanned may be used to automatically generate a coordinate. The coordinate may be a coordinate that is on or proximate to the preparation tooth being scanned in a 2D or 3D coordinate system. In some embodiments, a graphical user interface may include a hint feature, which may be a dot or point that a user may click on and move (e.g., via a drag and drop procedure) to select the coordinate. The coordinate of the hint feature (e.g., on-screen dot) may be the received coordinate in some embodiments.

At block 268, processing logic performs one or more morphological operations on the 3D surface to divide the 3D surface into a plurality of parts. In most cases, the plurality of parts corresponds to a plurality of distinct teeth. This is particularly true if the teeth have already been segmented in space (e.g., by a machine learning model trained to segment teeth from images and/or 3D surfaces). In some embodiments, prior to performance of the one or more morphological portions, the data for the 3D surface and/or the intraoral scans used to generate the 3D surface are input into a trained machine learning model which outputs tooth segmentation information. For example, the machine learning model may generate an output including multiple classifications, where one classification may correspond to soft tissue, and other classifications may each correspond to a different tooth. The tooth segmentation information together with a result of the one or more morphological operations may accurately divide the 3D surface into distinct teeth. Examples of morphological operations that may be performed include an erode operation and/or a dilate operation. The morphological operations may easily distinguish, for example, adjacent teeth that are separated by a thin “bridge” of connective soft and/or hard tissue.

At block 270, processing logic determines a first tooth of the plurality of teeth that is closest to the received coordinate (e.g., in the 3D coordinate space of the 3D surface). This may include finding a point on the 3D surface that is closest to the coordinate, and determining that the closest point is a point of a part associated with the first tooth. In some embodiments, a shape of the first tooth may be compared to one or more shape criteria, and if the shape fails to meet one or more of the shape criteria, a shape of the tooth may be modified. For example, if the final surface of the tooth is determined to be too elongated (e.g., is an elliptical shape in which a ratio between a first axis and a second axis of the ellipse exceeds a threshold), then excess surface may be removed from the sides of the tooth (e.g., the long sides of the ellipse to cause the ratio between the first axis and the second axis to be reduced).

At block 272, processing logic displays a view of the 3D surface. In embodiments, processing logic determines at least a portion of the first tooth to be displayed using a first visualization. If the closest tooth to the coordinate is a preparation tooth, then processing logic may use an identified margin line as a boundary for the preparation tooth. Processing logic may determine that the coordinate is within the margin line in a plane (e.g., an occlusal plane), and may classify points on the preparation tooth that are within the margin line as the preparation tooth. An area of the tooth within the margin line may be shown with the first visualization, while an area of the tooth outside of the margin line and other teeth and/or gingiva may be shown using a second visualization and/or one or more other visualizations. In one embodiment, at block 274 a first visualization is used for displaying the first tooth and at block 276 a second visualization is used for displaying a remainder of the teeth. The first visualization may be an opaque (e.g., 0% transparency) visualization, and the second visualization may be a semi-transparent (e.g., 50-99% transparency) visualization.

By displaying only the selected tooth using the first visualization (e.g., making only the selected tooth opaque and other teeth semi-transparent), mesial surfaces and distal surfaces that are proximate to adjacent teeth may be visible through the adjacent teeth due to the semi-transparent visualization of the adjacent teeth.

At block 278, processing logic may receive a new coordinate. For example, a user may select a hint feature displayed in the graphical user interface, and may drag the hint feature to a new location (causing the hint feature to have the new coordinate).

At block 280, processing logic determines a second tooth of the plurality of teeth that is closest to the new coordinate. At block 282, processing logic updates the view of the 3D surface. In particular, processing logic uses the second visualization for displaying the first tooth since it is no longer the selected tooth, and uses the first visualization for displaying the second tooth since it is now the selected tooth.

If the selected tooth is a preparation tooth, then processing logic may also display one or more sections of a margin line around the selected preparation tooth. The segments of the margin line may be displayed according to their assigned grades or scores. If the selected tooth is not a preparation tooth, then no margin line may be shown.

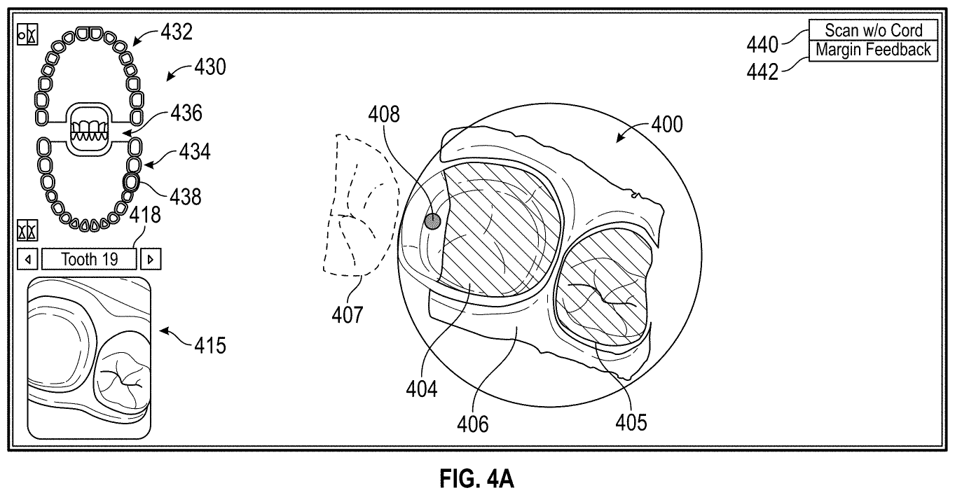

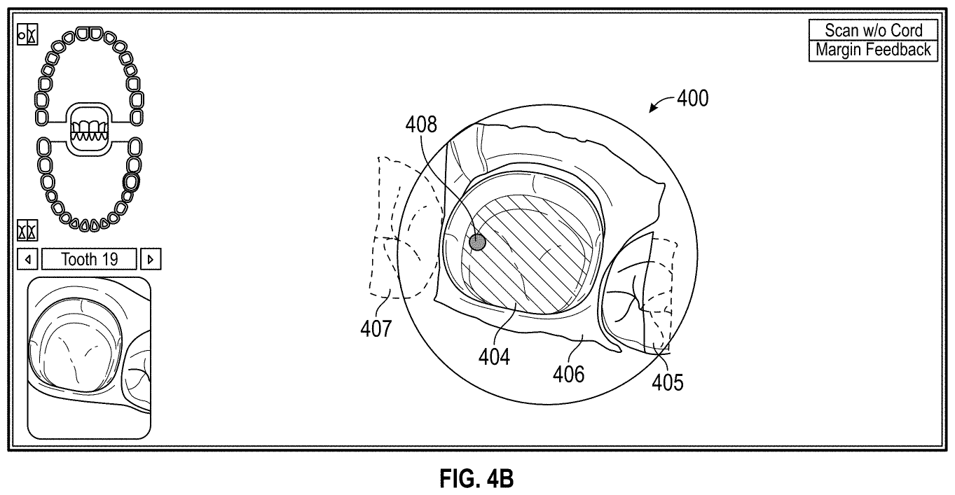

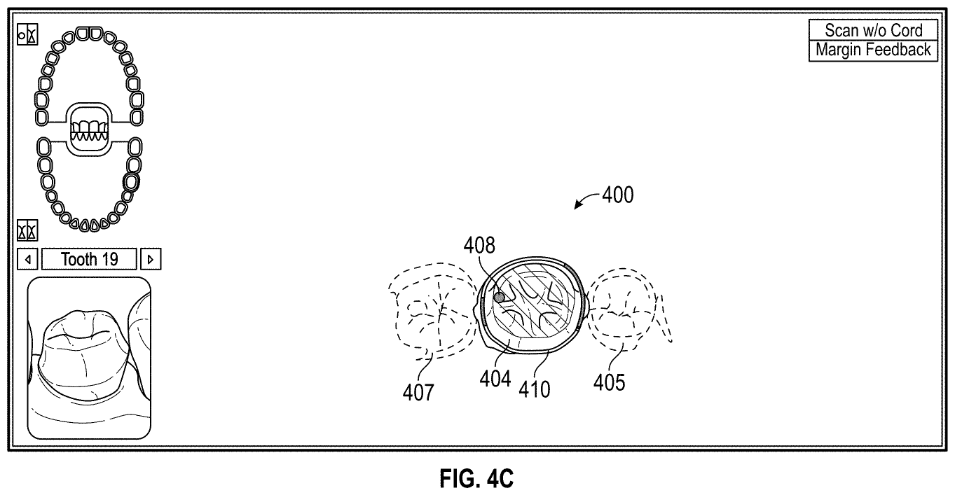

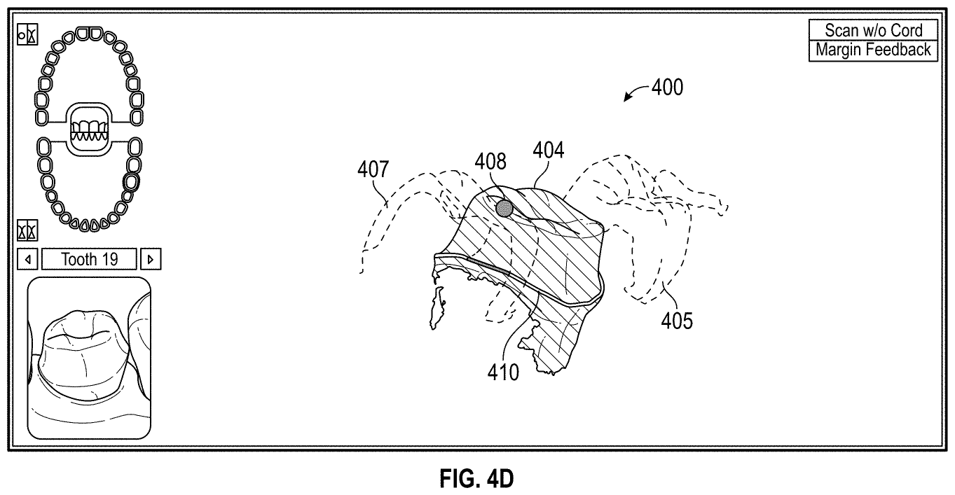

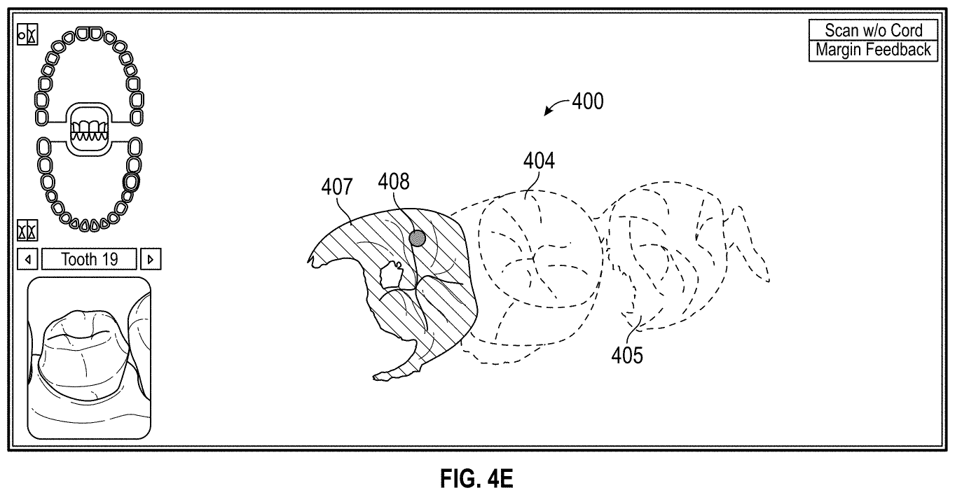

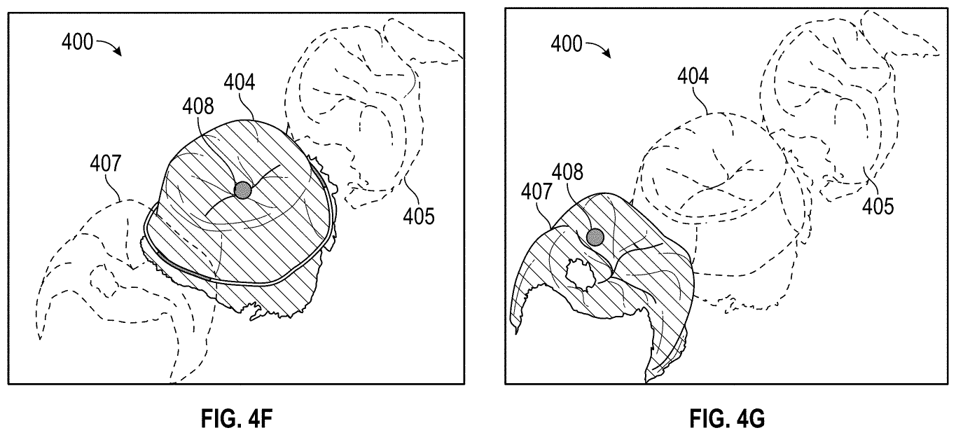

FIGS. 4A-G illustrate a graphical user interface (GUI) displaying views of a 3D surface 400 of a dental site generated from intraoral scan data during an intraoral scan session, in accordance with embodiments. The GUI further includes a viewfinder image 415 (e.g., a color viewfinder image) showing a current field of view (FOV) of an intraoral scanner and a scan segment indicator 430.

The 3D surface 400 is generated by registering and stitching together multiple intraoral scans captured during an intraoral scanning session. As each new intraoral scan is generated, that scan is registered to the 3D surface and then stitched to the 3D surface. Accordingly, the 3D surface becomes more and more accurate with each intraoral scan, until the 3D surface is complete. A 3D model may then be generated based on the intraoral scans.

In one embodiment, as shown, a scan segment indicator 430 may include an upper dental arch segment indicator 432, a lower dental arch segment indicator 434 and a bite segment indicator 436. While the upper dental arch is being scanned, the upper dental arch segment indicator 432 may be active (e.g., highlighted). Similarly, while the lower dental arch is being scanned, the lower dental arch segment indicator 434 may be active, and while a patient bite is being scanned, the bite segment indicator 436 may be active. Additionally, a user may select a particular tooth for a preparation tooth scan segment 438. The GUI shows that tooth 19 has been selected in tooth indicator 418 for a preparation tooth scan segment 438. A user may select a particular segment indicator 432, 434, 436, 438 to cause a 3D surface associated with a selected segment to be displayed. A user may also select a particular segment indicator 432, 434, 436, 438 to indicate that scanning of that particular segment is to be performed. Alternatively, processing logic may automatically determine a segment being scanned, and may automatically select that segment to make it active.

The GUI may further include a task bar with multiple modes of operation or phases of intraoral scanning. Selection of a patient selection mode may enable a doctor to input patient information and/or select a patient already entered into the system. Selection of a scanning mode enables intraoral scanning of the patient's oral cavity. After scanning is complete, selection of a post processing mode may prompt the intraoral scan application to generate one or more 3D models based on intraoral scans and/or 2D images generated during intraoral scanning, and to optionally perform an analysis of the 3D model(s). Examples of analyses that may be performed include analyses to detect areas of interest, to assess a quality of the 3D model(s), and so on. Once the doctor is satisfied with the 3D models, they may generate orthodontic and/or prosthodontic prescriptions. Selection of a prescription fulfillment mode may cause the generated orthodontic and/or prosthodontic prescriptions to be sent to a lab or other facility to cause a prosthodontic device (e.g., a crown, bridge, denture, etc.) or orthodontic device(e.g., an orthodontic aligner) to be generated.

In embodiments, the GUI may include a button and/or indicator 440 showing whether a preparation scanning mode and/or partial retraction scanning technique are selected. A user may select or click on the button and/or indicator 440 to manually enable or disable preparation scanning mode and/or a partial retraction scanning mode. Additionally, or alternatively, the GUI may include a button and/or indicator 441 showing whether margin line feedback is active. A user may enable or disable margin line feedback (e.g., in which margin line segments are graded and shown in the GUI) by selecting or clicking on the button and/or indicator 441.

As shown in FIG. 4A, when scans are initially generated (e.g., of a preparation scan segment) gingiva 406, a first tooth 404 (e.g., a preparation tooth), a second tooth 405 and a third tooth 407 may all be shown using a semi-transparent visualization. Also shown is a hint feature 408, which may be a dot or other shape that a user can drag to any desired location relative to the 3D surface 400. The hint feature 408 may be used to determine a current tooth under consideration.

FIG. 4B shows 3D surface 400 after processing has been performed to identify hard and soft tissue and to determine that first tooth 404 is a closest tooth to the hint feature 408. After determining that first tooth 404 is the closest tooth to hint feature 408, first tooth is shown with an opaque visualization. Second tooth 405, third tooth 407 and gingiva 406 are still shown using a semi-transparent visualization.

A user may select to have the soft tissue (e.g., gingiva) be visible (e.g., semi-transparent) or invisible (e.g., 100% transparent). FIG. 4C shows 3D surface 400 after soft tissue has been classified and then rendered invisible. FIG. 4C also shows margin feedback 410 in the form of multiple graded and color coded margin line segments.

The 3D surface 400 can be checked visually by the doctor. The doctor can virtually manipulate the 3D surface via the user interface with respect to up to six degrees of freedom (i.e., translated and/or rotated with respect to one or more of three mutually orthogonal axes) using suitable user controls (hardware and/or virtual) to enable viewing of the 3D surface from any desired direction. This can include, for example, zooming in, zooming out, panning, rotating, and so on. Accordingly, the doctor may review (e.g., visually inspect) the generated 3D surface of a dental site and determine whether the 3D surface is acceptable (e.g., whether a margin line of a preparation tooth is accurately represented in the 3D surface).

FIG. 4D shows the 3D surface 400 after a user has rotated and zoomed in on the 3D surface 400. As shown, because the first tooth 404 is selected (e.g., based on being a closest tooth to the hint feature 408), first tooth is shown with an opaque visualization and second tooth 405 and third tooth 407 are shown with a semi-transparent visualization. In this example, gingiva are invisible. Accordingly, a user can view the mesial and distal portions of the first tooth 404, including the mesial and distal regions of the margin line 410 through the semi-transparent views of the first tooth 405 and/or second tooth 407.

FIG. 4E shows 3D surface 400 after the view has been further rotated, and after the hint feature 408 has been dragged to third tooth 407. Accordingly, third tooth 407 is now the closest tooth to hint feature 408. As a result, third tooth 407 is shown with an opaque visualization and first tooth 404 is shown with a semi-transparent visualization. Since third tooth 407 is not a preparation tooth, no margin line is shown for third tooth 407.

FIGS. 4F-G show the same zoom and orientation of the 3D surface 400, except that first tooth 408 is a closest tooth to the hint feature 408 in FIG. 4F and third tooth 407 is a closest tooth to hint feature 408 in FIG. 4G. Accordingly, first tooth 404 is opaque in FIG. 4F, while third tooth 407 is opaque in FIG. 4G.

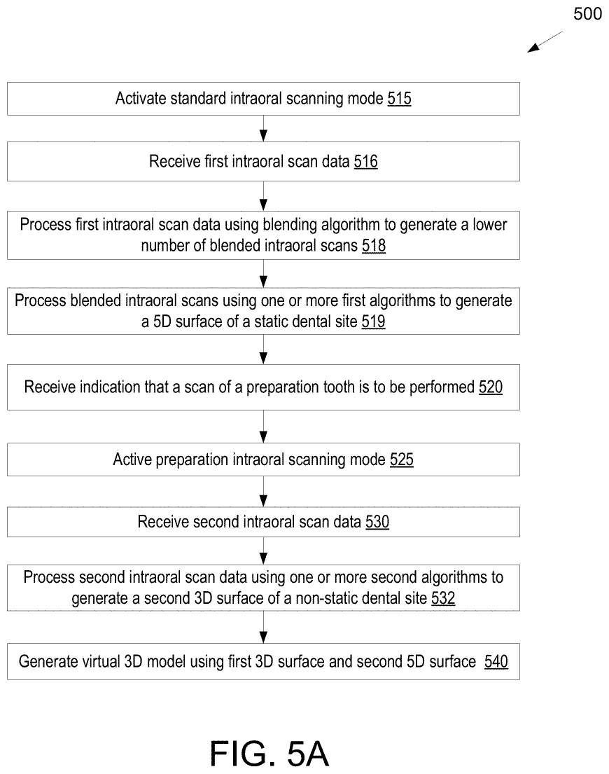

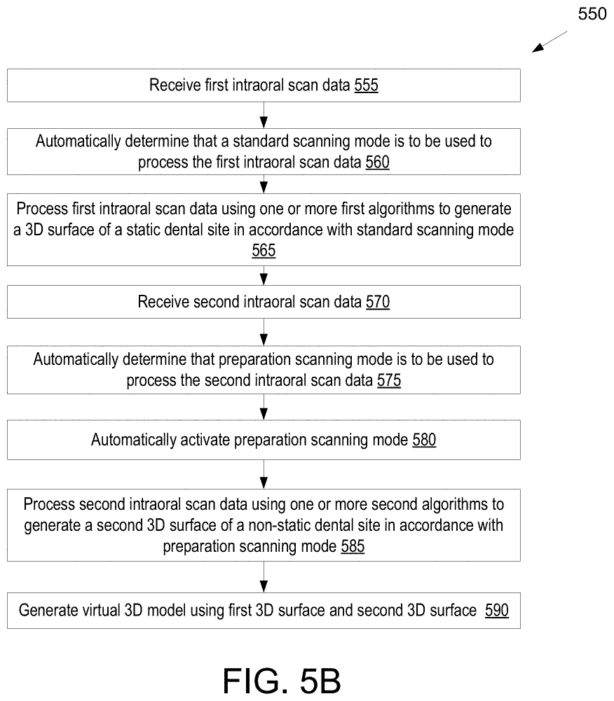

FIG. 5A illustrates a flow diagram for a method 500 of scanning a dental site, in accordance with an embodiment. At block 515 of method 500, processing logic activates a first intraoral scanning mode. The first intraoral scanning mode may be, for example, a default intraoral scanning mode that is activated automatically. For example, the standard scanning mode may be a default intraoral scanning mode, and may be activated automatically. Alternatively, a user input may be received selecting the first intraoral scanning mode.

At block 516, processing logic receives first intraoral scan data. The first intraoral scan data may include a sequence of intraoral scans, which may be consecutive and/or raw intraoral scans. The first intraoral scan data may be received during an intraoral scanning session shortly after the first intraoral scan data was generated by an intraoral scanner and while the intraoral scanner continues to generate additional intraoral scan data in embodiments.

At block 518, processing logic processes the first intraoral scan data using a blending algorithm to generate a lower number of blended intraoral scans. At block 519, processing logic processes the blended intraoral scans using one or more first algorithms to generate a 3D surface of a static dental site. The dental site may be static in the sense that no clinically significant changes occur at the dental site within a minimum time period (e.g., within a 30 second time period, within a 1 minute time period, within a 2 minute time period, etc.). The one or more first algorithms may correspond, for example, to the first algorithms of standard scanning module 119 described with reference to FIG. 1. The first dental site may be or include, for example, an upper dental arch and/or a lower dental arch of a patient. The first dental site (e.g., one of the dental arches) may include a natural tooth thereon (e.g., a tooth other than a preparation tooth). For example, a doctor may have scanned all of the dental site (e.g., the dental arch) except for a preparation tooth.

At block 520, processing logic receives an indication that a preparation tooth is to be scanned, and/or processing logic otherwise determines that a preparation tooth is to be scanned or is being scanned. The indication may or may not identify a specific preparation tooth that is to be scanned. If the specific preparation tooth is not identified by the doctor, then processing logic may later automatically identify the preparation tooth based on the shape of the preparation tooth and/or surrounding surfaces around the preparation tooth (e.g., adjacent teeth). In alternative embodiments, an indication that a preparation tooth was scanned may be received after receiving the second intraoral scan data. In further embodiments, processing logic may automatically determine that a preparation tooth was scanned based on analysis of second intraoral scan data received at block 530 (and the preparation scanning mode may be activated after receiving and analyzing the second intraoral scan data).

At block 525, processing logic activates a preparation scanning mode. At block 530, processing logic receives second intraoral scan data. The second intraoral scan data may be a sequence of intraoral scans of the preparation tooth. The sequence of intraoral scans may have been generated while a doctor performs a partial retraction intraoral scanning technique as discussed herein above. For example, the doctor may expose just a small portion of the margin line for the preparation tooth at a time by using a tool to retract a small region of the gingiva around the preparation tooth, and may generate one or more scans of that small portion of the margin line and surrounding surfaces while that small portion of the margin line is exposed. The doctor then moves the tool, exposes a new portion of the margin line by retracting a new region of the gingiva, and generates one or more additional scan of the newly exposed portion of the margin line. The doctor continues this process until scans are generated for all portions of the margin line.

At block 532, processing logic processes the second intraoral scan data using one or more second algorithms to generate a second 3D surface of a non-static dental site. In this example, the non-static dental site is the preparation tooth with the exposed margin line and gingiva around the preparation tooth. In one embodiment, a different portion of the margin line is exposed and a different portion of the gingiva is retracted in each set of intraoral scans included in the second intraoral scan data, and thus the preparation tooth is considered to be a non-static dental site. In one embodiment, the preparation tooth includes collapsing gums that are collapsing around the margin line of the preparation tooth, and may be considered a non-static dental site. Alternatively, such a situation may be considered a static dental site if the gums are collapsing slowly enough. The first algorithms used to process the first intraoral scan data at block 519 may not generate an accurate depiction of the margin line due in part to the non-static nature of the gingiva and margin line received at block 530. Accordingly, the one or more second algorithms are used at block 532, which may be configured to operate on scan data of a non-static dental site. The one or more second algorithms may correspond, for example, to the second algorithms of preparation scanning logic 118 described with reference to FIG. 1.

In one embodiment, no blending algorithm is used to generate blended scans for the preparation scanning mode. Alternatively, blended scans may be generated for the partial retraction intraoral scanning mode. In embodiments, a moving tissue detection algorithm that is used is more aggressive than a moving tissue detection algorithm of the standard scanning mode. This may cause the moving tissue detection algorithm of the preparation scanning mode to classify regions as moving tissue that would not be classified as moving tissue by the moving tissue detection algorithm of the standard scanning mode. The moving tissue detection algorithms of the standard scanning mode are generally ill suited to detecting moving tissue for preparation teeth because preparation teeth are generally associated with changing gingiva, excess bleeding, excess saliva, etc. as compared to standard teeth, for which moving tissue is generally limited to tongue, dental tools and cheeks, for example. Accordingly, a unique modified moving tissue detection algorithm has been found to work better for clean-up of intraoral scans of preparation teeth. The unique modified moving tissue detection algorithm has different thresholds for identifying moving tissue than the standard moving tissue detection algorithm. For example, the unique moving tissue detection algorithm may have a lower size threshold for moving tissue and may identify smaller regions as moving tissue than the standard moving tissue detection algorithm.

In embodiments, the unique moving tissue detection algorithm used for the preparation scanning mode uses a protection based on region classifications (e.g., as determined by a neural network). In embodiments, areas classified as hard tissue (e.g., as teeth) are not removed by the moving tissue detection algorithm.

In embodiments, a trained machine learning model classifies margin line areas, as discussed in greater detail elsewhere herein. The area classified as a margin line area is much smaller than the whole surface of the processed scans and/or 3D surface, and allows the use of more accurate and/or time consuming algorithms than algorithms (e.g., machine learning models) trained to classify hard tissue and soft tissue.

In some embodiments, artificial directions are used to find conflicts. Examples of directions that may be used include a center of mass direction, an orthogonal to insertion path direction, and so on. For example, a center of mass may be determined, and a vector may be determined between a point or pixel and the center of mass. A direction of that vector may then be used to find conflict. In another example, an insertion path may be computed for inserting a cap, bridge or other prosthodontic onto a preparation tooth. A direction orthogonal to the insertion path may then be determined and used to find conflict. In embodiments, surface points can be projected along the determined directions and treated as moving tissue if there are other parts of the surface along such directions on a projection ray.

In one embodiment, processing the second intraoral scan data (which may include a plurality of intraoral scans) using the one or more second algorithms includes determining a conflicting surface for a pair of intraoral scans from the second intraoral scan data. This may be performed as part of a merging algorithm. A first intraoral scan of the pair of intraoral scans may have a first distance from a probe of an intraoral scanner for the conflicting surface and a second intraoral scan of the pair of intraoral scans may have a second distance from the probe of the intraoral scanner for the conflicting surface. Processing logic may then determine which of the distances is greater. For example, processing logic may determine that the first distance is greater than the second distance. Processing logic may additionally determine a difference between the two distances, and determine whether the difference between the first distance and the second distance is greater than a difference threshold. Responsive to determining that the difference is greater than the difference threshold, processing logic discards a representation of the conflicting surface from the intraoral scan with the smaller distance (e.g., from the first intraoral scan in the above example). The second 3D surface of the non-static dental site (e.g., of the preparation tooth) may then be determined by combining data from the first intraoral scan and the second intraoral scan, wherein the discarded representation of the conflicting surface from the first intraoral scan may not be used for the surface. If the difference is less than the difference threshold, then the data for the conflicting surface from the two intraoral scans may be averaged together. The difference threshold used for the moving tissue detection algorithm of the preparation scanning mode may be lower than the difference threshold used for the standard scanning mode. Accordingly, the preparation scanning mode's moving tissue detection algorithm may identify regions of scans with certain differences as moving tissue which would be too small a difference for the standard scanning mode's moving tissue detection algorithm to identify them as moving tissue.

In one embodiment, processing the second intraoral scan data using the one or more algorithms configured to determine a three-dimensional surface of a non-static dental site further comprises determining a conflicting surface for a pair of intraoral scans from the second intraoral scan data. This may be performed as part of a merging algorithm. Processing logic may determine a first mean curvature or Gaussian curvature for the conflicting surface from a first intraoral scan from the pair. Processing logic may additionally determine a second mean curvature or Gaussian curvature for the conflicting surface from a second intraoral scan from the pair. Processing logic may then determine which of the mean curvatures is greater (e.g., processing logic may determine that the second mean curvature is less than the first mean curvature). Processing logic may additionally determine a difference between the two mean or Gaussian curvatures, and determine whether the difference between the first mean or Gaussian curvature and the second mean or Gaussian curvature is greater than a difference threshold. Responsive to determining that the difference is greater than the difference threshold, processing logic discards a representation of the conflicting surface from the intraoral scan with the smaller mean or Gaussian curvature (e.g., from the first intraoral scan in the above example). The second 3D surface of the non-static dental site (e.g., of the preparation tooth) may then be determined by combining data from the first intraoral scan and the second intraoral scan, wherein the discarded representation of the conflicting surface from the first intraoral scan is not used to determine the surface. If the difference is less than the difference threshold, then the data for the conflicting surface from the two intraoral scans may be averaged together.

In some embodiments, both the distances and mean or Gaussian curvatures are determined for conflicting surfaces, and these values are used together to determine which scan data to discard. In one embodiment, conflicting surface data is averaged together if both the difference between distances is below a distance difference threshold and the difference between mean curvatures or Gaussian curvatures is below a curvature difference threshold. If either the distance difference is greater than the distance difference threshold or the mean or Gaussian curvature is greater than the curvature difference threshold, then the associated data from one of the two scans is discarded as described above.

It should be noted that though regions identified as moving tissue are discussed as being discarded, that data associated with these regions may in actuality not be discarded. The data may be filtered out such that it is not used for the 3D surface. However, the data may still be available for future processing, such as if later processing indicates that there is a void or hole at an area for which data was previously discarded. Under such a circumstance, the data that was “discarded” may be added back and used for the surface, such as to fill a void.

At block 540, processing logic generates a virtual 3D model using the first 3D surface of the static dental site determined from the first intraoral scan data and the second 3D surface of the non-static dental site determined from the second intraoral scan data. In instances where the static dental site includes the non-static dental site (e.g., the static dental site is a dental arch and the non-static dental site is a preparation tooth on the dental arch), the portion of the 3D surface of the dental site that depicts the non-static dental site may be erased or omitted and replaced by the 3D surface of the non-static dental site.

FIG. 5B illustrates a flow diagram for a method 550 of using two different scanning modes for scanning a preparation tooth, in accordance with an embodiment. At block 555 of method 550, processing logic receives first intraoral scan data. At block 560, processing logic automatically determines that a first scanning mode is to be used to process the first intraoral scan data. For example, processing logic may determine based on analysis of the first intraoral scan data that it does not represent a preparation tooth, and that a standard intraoral scanning mode is to be used to process the first intraoral scan data. At block 565, processing logic processes the first intraoral scan data using one or more first algorithms to generate a 3D surface of a static dental site (e.g., of a dental arch or a portion of a dental arch) in accordance with the first scanning mode.

At block 570, processing logic receives second intraoral scan data. At block 575, processing logic automatically determines that a second scanning mode (e.g., the preparation scanning mode) is to be used to process the second intraoral scan data. For example, processing logic may determine based on analysis of the second intraoral scan data that it was generated using a partial retraction scanning technique, and that a preparation scanning mode is to be used to process the second intraoral scan data. Such a determination may be made, for example, based on comparison of intraoral images (e.g., sequentially generated images) from the second intraoral scan data and determining that differences there between exceed a threshold. The differences may be compared to one or more thresholds, and if a threshold percentage of the differences exceed a difference threshold, then processing logic may determine that the second intraoral scanning mode is to be used. At block 580, processing logic may automatically activate the second scanning mode (e.g., the preparation scanning mode). At block 585, processing logic processes the second intraoral scan data using one or more second algorithms to generate a 3D surface of a non-static dental site (e.g., of a preparation tooth) in accordance with the second scanning mode (e.g., the partial retraction scanning mode). At block 590, processing logic generates a virtual 3D model using the first 3D surface and the second 3D surface.

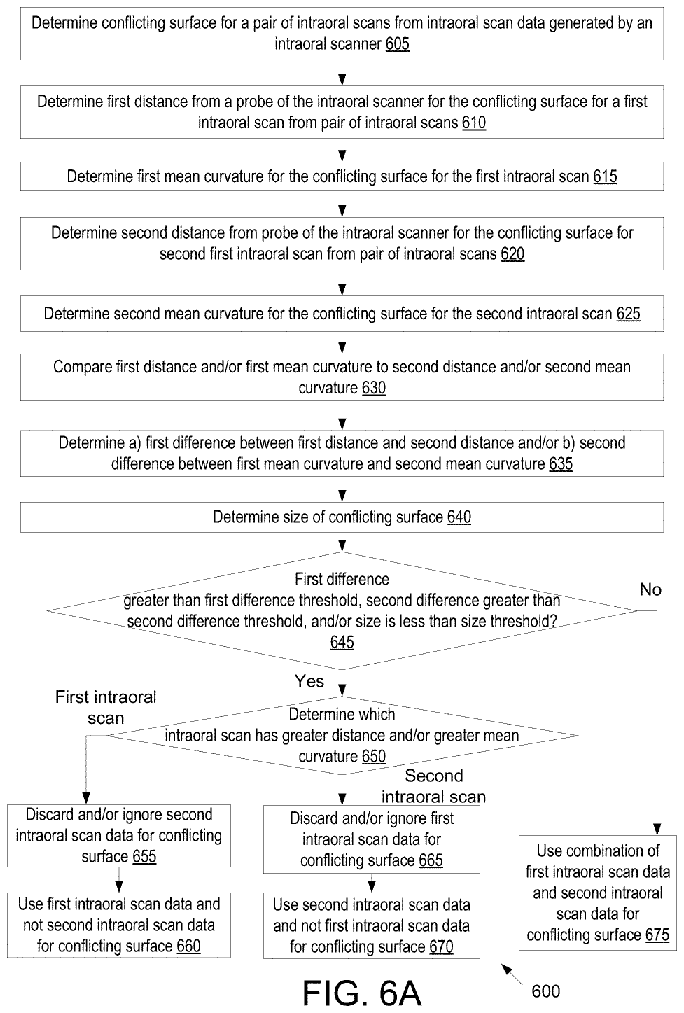

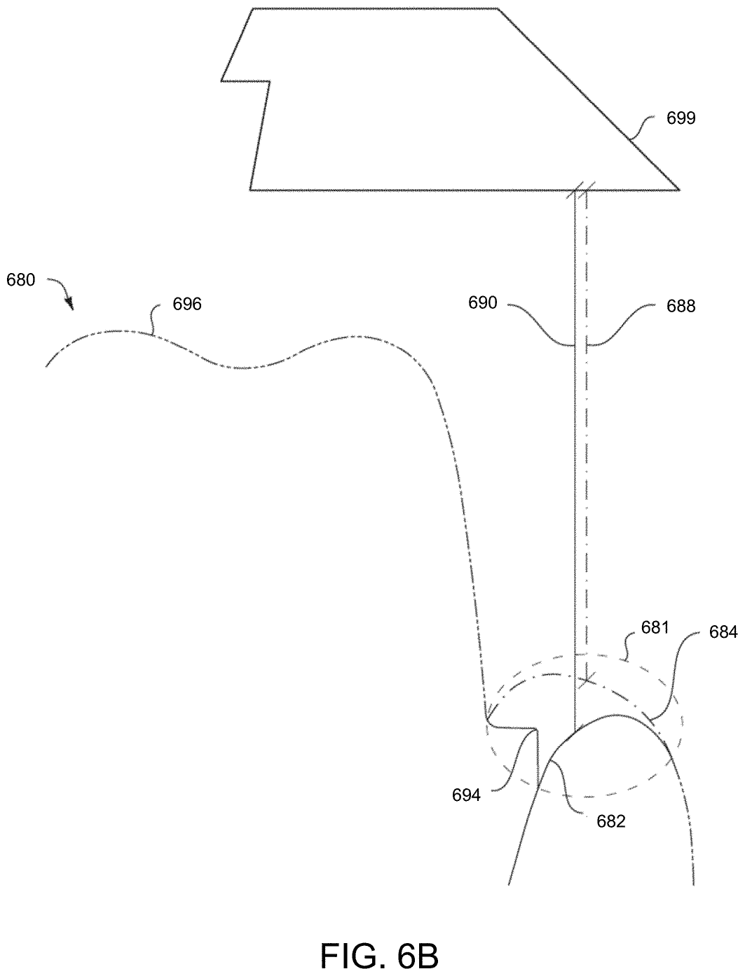

FIG. 6A illustrates a flow diagram for a method 600 of resolving conflicting scan data of a dental site, in accordance with an embodiment. Method 600 may be performed by the one or more second algorithms of preparation scanning logic 118 of FIG. 1 to select which scan data to keep and which scan data to discard for conflicting surfaces. In one embodiment, the preparation scanning logic 118 performs method 600 as part of a stitching and/or merging algorithm used to generate a 3D surface from a plurality of intraoral scans. In one embodiment, processing logic determines that a partial retraction scan of a first preparation tooth will be performed or has been performed, wherein the partial retraction scan comprises an intraoral scan of a preparation tooth that has not been packed with a gingival retraction cord. Processing logic receives a plurality of intraoral scans generated by an intraoral scanner (before or after making the determination that the partial retraction scanning technique was performed), and processes the plurality of intraoral scans using a stitching or merging algorithm to stitch together the plurality of intraoral scans in accordance with a partial retraction intraoral scanning mode. In one embodiment, the stitching algorithm (also referred to as a merging algorithm) executes method 600.

At block 605 of method 600, processing logic determines a conflicting surface for a pair of intraoral scans from intraoral scan data generated by an intraoral scanner. At block 610, processing logic determines a first distance from a probe of the intraoral scanner (also referred to as a first depth and/or first height) for the conflicting surface for a first intraoral scan of the pair of intraoral scans. The first depth may be a combined depth value (e.g., an average depth or media depth) based on the depths of some or all pixels of the first intraoral scan that are included in the conflicting surface. At block 615, processing logic determines a first mean curvature (or a first Gaussian curvature) for the conflicting surface for the first intraoral scan. At block 620, processing logic determines a second distance from the probe of the intraoral scanner (also referred to as a second depth and/or second height) for the conflicting surface for a second intraoral scan of the pair of intraoral scans. The second depth may be a combined depth value (e.g., an average depth or media depth) based on the depths of some or all pixels of the second intraoral scan that are included in the conflicting surface. At block 625, processing logic determines a second mean curvature (or a second Gaussian curvature) for the conflicting surface for the second intraoral scan.

At block 630, processing logic compares the first distance and/or the first mean curvature (or first Gaussian curvature) to the second distance and/or the second mean curvature (or second Gaussian curvature). At block 635, processing logic determines a) a first difference between the first distance and the second distance and/or b) a second difference between the first mean curvature (or second Gaussian curvature) and the second mean curvature (or second Gaussian curvature). At block 640, processing logic determines a size of the conflicting surface.

At block 645, processing logic determines one or more of the following: a) whether the first difference is greater than a first difference threshold, b) whether the second difference is greater than a second difference threshold, c) whether the size of the conflicting surface is less than an upper size threshold. Processing logic may also determine whether the size is greater than a lower size threshold. If the first difference is less than the first difference threshold, the second difference is less than the second difference threshold, the size is less than the upper size threshold, and/or the size is greater than the lower size threshold, then the method proceeds to block 675. In one embodiment, the method proceeds to block 675 if the first difference is greater than the first difference threshold and the size of the conflicting surface is within a particular size range (e.g., smaller than an upper size threshold and/or larger than a lower size threshold). In one embodiment, the method proceeds to block 675 if the first difference is greater than the first difference threshold, the second difference is greater than the second difference threshold, and the size of the conflicting surface is within a particular size range. Otherwise, the method continues to block 650.

At block 675, processing logic uses a combination of the first intraoral scan data and the second intraoral scan data for the conflicting surface. This may include, for example, averaging the first intraoral scan data with the second intraoral scan data for the conflicting surface. The first and second intraoral scan data may be averaged with a weighted or non-weighted average. For example, the intraoral scan with the greater distance measurement (e.g., greater height measurement or lesser depth measurement) may be assigned a higher weight than the intraoral scan with the lesser distance measurement (e.g., lesser height measurement or greater depth measurement).

At block 650, processing logic determines which of the intraoral scans has a greater distance and/or a greater mean curvature (or greater Gaussian curvature) for the conflicting surface. If the first intraoral scan has a greater distance and/or a greater mean curvature than the second intraoral scan for the conflicting surface, then the method continues to block 655. If the second intraoral scan has a greater distance and/or a greater mean curvature than the first intraoral scan for the conflicting surface, then the method continues to block 665.

At block 655, processing logic discards and/or ignores the second intraoral scan data for the conflicting surface. At block 660, processing logic uses the first intraoral scan data and not the second intraoral scan data for the conflicting surface when generating a 3D surface for a virtual 3D model of the dental site that was scanned.

At block 665, processing logic discards and/or ignores the first intraoral scan data for the conflicting surface. At block 670, processing logic uses the second intraoral scan data and not the first intraoral scan data for the conflicting surface when generating the 3D surface for the virtual 3D model of the dental site that was scanned.

FIG. 6B illustrates resolution of conflicting scan data of a dental site that includes a preparation tooth and surrounding gingiva, in accordance with an embodiment. In FIG. 6B, a dental site 680 is scanned by an intraoral scanner. The conflicting scan data includes data of a first scan that was taken while a margin line 694 was exposed and data of a second scan that was taken while the margin line 694 was covered by gingiva. The first scan shows surfaces 682, 696, which includes exposed margin line 694. The second scan shows surfaces 696, 684, which includes gingiva that overlies the margin line. The margin line is not detected in the second scan. A conflicting surface 681 may be determined based on comparison between the two scans.

For the first scan, a first distance 690 from the probe is determined for the conflicting surface 681. The first distance may be an average distance from the probe for surface 682 in one embodiment. However, a first distance 690 for a particular point on surface 682 is illustrated for the purposes of clarity. For the second scan, a second distance 688 from the probe is determined for surface 684. The second distance may be an average distance of the second scan for the surface 684. However, a second distance 688 for a particular point is illustrated for the purposes of clarity. As described with reference to FIG. 6A, the first and second distances may be compared, and a difference between these distances may be computed. Processing logic may determine that the first difference is greater than a difference threshold, and that the first distance 690 is greater than the second distance 688. The second scan data for the conflicting surface 681 may then be discarded so that the 3D model that is generated depicts the margin line 694.

A first mean curvature may also be computed for the first surface 682 of the conflicting surface 681 and a second mean curvature may be computed for the second surface 684 of the conflicting surface 681. As shown, the first surface 682 would have a greater mean curvature than the second surface 684. The first and second mean curvatures may be compared, and the result of the comparison may be used as an additional data point to determine which of the scans should be used to depict the conflicting surface, as described with reference to FIG. 6A.

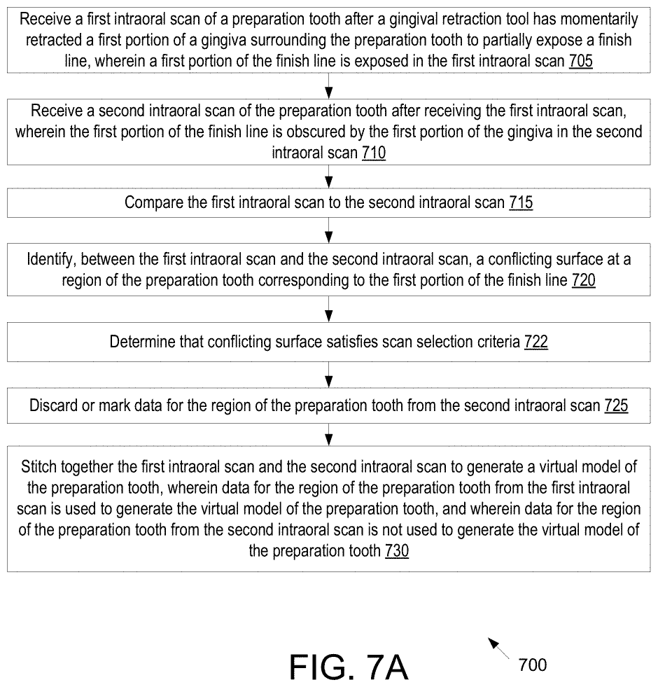

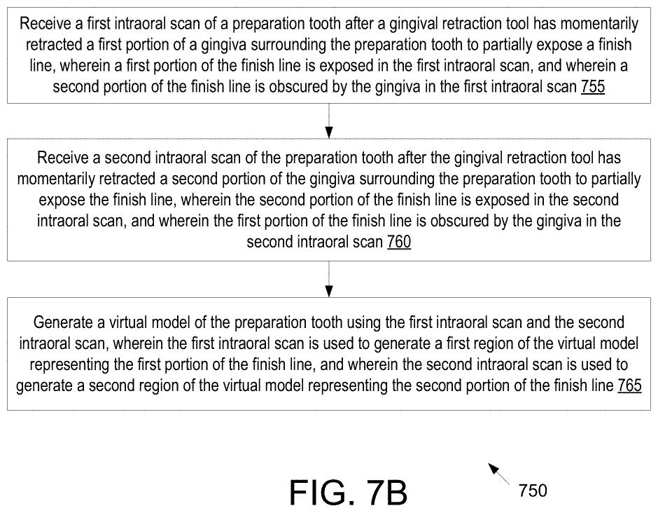

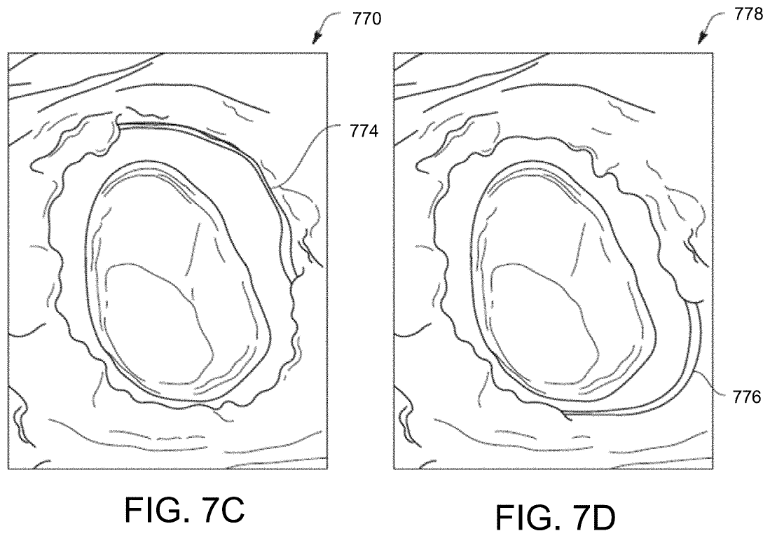

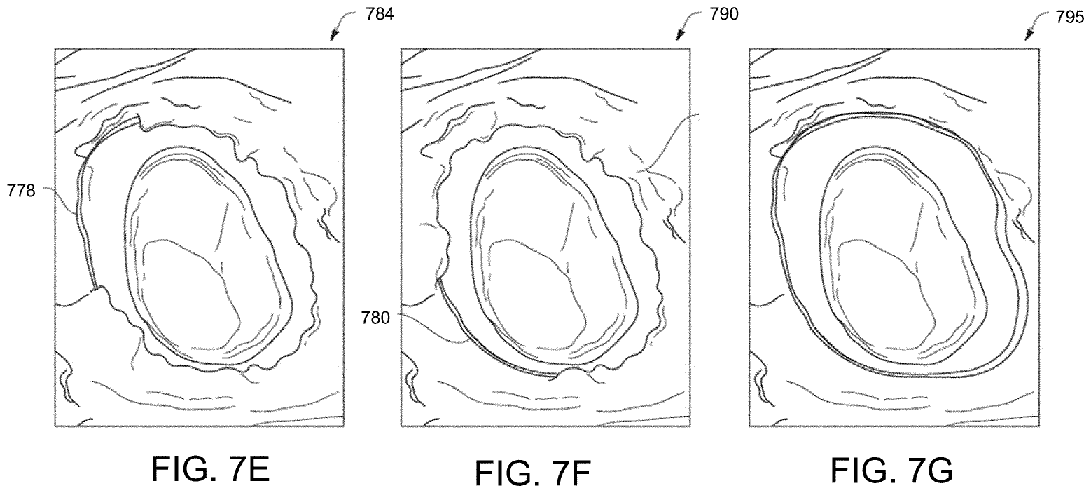

FIG. 7A illustrates a flow diagram for a partial retraction method 700 of scanning a preparation tooth, in accordance with an embodiment. At block 705 of method 700, processing logic receives a first intraoral scan of a preparation tooth after a gingival retraction tool has momentarily retracted a first portion of a gingiva surrounding the preparation tooth to partially expose a margin line. The first portion of the margin line is exposed in the first intraoral scan. At block 710, processing logic receives a second intraoral scan of the preparation tooth after receiving the first intraoral scan. In the second intraoral scan, the first portion of the margin line is obscured by the first portion of the gingiva. For example, the gingival retraction tool may have been moved to expose a different portion of the margin line, letting the gingiva collapse back over the first portion of the margin line when the second intraoral scan was generated.

At block 715, processing logic compares the first intraoral scan to the second intraoral scan. At block 720, processing logic identifies, between the first intraoral scan and the second intraoral scan, a conflicting surface at a region of the preparation tooth corresponding to the first portion of the margin line. At block 722, processing logic determines that the conflicting surface satisfies scan selection criteria. The scan selection criteria may include, for example, any of the criteria described with reference to FIGS. 6A-6B. For example, the scan selection criteria may include a first criterion that an average distance difference between the scans for the conflicting surface area be greater than a distance difference threshold. The scan selection criteria may further include a second criterion that the scan with the larger average distance be selected. The scan selection criteria may further include a third criterion that the scan with the larger mean curvature be selected. Other criteria may also be used.

At block 725, processing logic discards or marks data for the region of the preparation tooth associated with the conflicting surface from the second intraoral scan. At block 730, processing logic stitches together the first intraoral scan and the second intraoral scan to generate a virtual model of the preparation tooth. Data for the region of the preparation tooth from the first intraoral scan is used to generate the virtual model of the preparation tooth, and data for the region of the preparation tooth from the second intraoral scan is not used to generate the virtual model of the preparation tooth.

FIG. 7B illustrates another flow diagram for a partial retraction method 750 of scanning a preparation tooth, in accordance with an embodiment. In one embodiment, method 750 is performed after receiving an indication that a partial retraction scan will be performed, and activating a partial retraction intraoral scanning mode.

At block 755 of method 750, processing logic receives a first intraoral scan of a preparation tooth after a gingival retraction tool has momentarily retracted a first portion of a gingiva surrounding the preparation tooth to partially expose a margin line, wherein a first portion of the margin line is exposed in the first intraoral scan, and wherein a second portion of the margin line is obscured by the gingiva in the first intraoral scan. At block 760, processing logic receives a second intraoral scan of the preparation tooth after the gingival retraction tool has momentarily retracted a second portion of the gingiva surrounding the preparation tooth to partially expose the margin line, wherein the second portion of the margin line is exposed in the second intraoral scan, and wherein the first portion of the margin line is obscured by the gingiva in the second intraoral scan. At block 765, processing logic generates a virtual model of the preparation tooth using the first intraoral scan and the second intraoral scan, wherein the first intraoral scan is used to generate a first region of the virtual model representing the first portion of the margin line, and wherein the second intraoral scan is used to generate a second region of the virtual model representing the second portion of the margin line. In one embodiment, a third portion of the margin line is exposed in the first intraoral scan and in the second intraoral scan, and both the first intraoral scan and the second intraoral scan are used to generate a third region of the virtual model representing the third portion of the margin line.

FIGS. 7C-G illustrate a partial retraction method of scanning a preparation tooth, in accordance with an embodiment. FIG. 7C illustrates a first view of a preparation tooth as depicted in first intraoral scan data 770 generated while a first region 774 of a margin line was exposed. FIG. 7D illustrates a second view of the preparation tooth as depicted in second intraoral scan data 778 generated while a second region 776 of the margin line was exposed. FIG. 7E illustrates a third view of the preparation tooth as depicted in third intraoral scan data 784 generated while a third region 778 of the margin line was exposed. FIG. 7F illustrates a fourth view of the preparation tooth as depicted in fourth intraoral scan data 790 generated while a fourth region 780 of the margin line was exposed. FIG. 7G illustrates a 3D model 795 of the preparation tooth generated using selected portions of first intraoral scan data 770, second intraoral scan data 778, third intraoral scan data 784 and fourth intraoral scan data 790. The selected portions are those respective portions showing the exposed margin line in each of the first intraoral scan data 770, second intraoral scan data 778, third intraoral scan data 784 and fourth intraoral scan data 790.

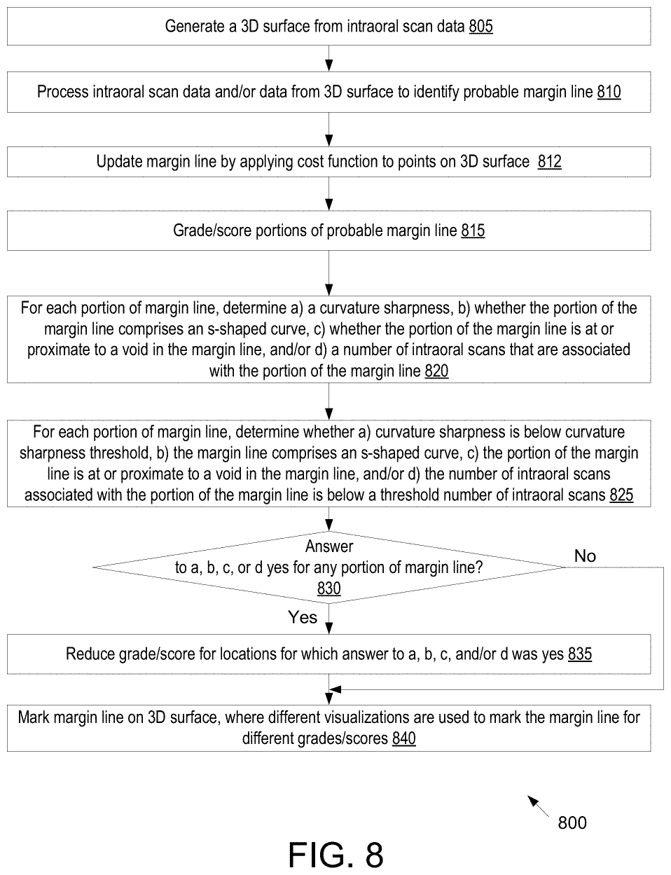

FIG. 8 illustrates a flow diagram for a method 800 of identifying and grading a margin line on a 3D surface of a preparation tooth, in accordance with an embodiment. At block 805 of method 800, a 3D surface of a preparation tooth is generated from intraoral scan data including multiple intraoral scans of the preparation tooth.

At block 810, the intraoral scan data and/or data from the 3D surface is processed to identify a probably margin line for the preparation tooth on the 3D surface. In one embodiment, data from the 3D surface is processed by projecting the 3D surface onto one or more 2D planes to generate images of the 3D surface. In one embodiment, the intraoral scan data and/or data from the 3D surface is processed by a machine learning model that has been trained to identify margin lines on preparation teeth. The machine learning model may output a probability map that indicates, for each surface point or patch (e.g., group of surface points) of the data input into the machine learning model, a probability that the surface point represents a margin line. In the case of scans or projections onto 2D surfaces, the probability map may then be projected back onto the 3D model or 3D surface to assign probability values to points on the 3D model or 3D surface. In embodiments, for each surface point, a probability of that surface point being classified as a margin line and a confidence of the surface point being classified as a margin line is determined.

In one embodiment, at block 812 a cost function may be applied to find the margin line using the probability values and/or confidence values assigned to the points on the 3D model or 3D surface. In one embodiment, processing logic generates a matrix that identifies, for each point (e.g., edge, vertex, voxel, etc. on a surface of the 3D model), a probability that the point represents a margin line. For example, entries in the matrix that have no chance of representing the margin line have an assigned 0% probability.

Processing logic may use the cost function to create a closest contour going through points with high probabilities of representing the margin line. In one embodiment, a total cost of the contour that is drawn for the margin line is the sum of all edges (e.g., vertexes) included in the margin line, adjusted by weights associated with each of the vertexes. Each weight for a vertex may be a function of the probability assigned to that vertex. The cost for that vertex being included in the margin line may be approximately 1/(A+FP), where A is a small constant and P is the probability of the vertex representing the margin line. The smaller the probability for a vertex, the larger the cost of that vertex being included in the margin line. Costs may also be computed for segments of the margin line based on a sum of the costs of the vertexes included those segments. When probability is close to 100%, then cost is approximately 1 adjusted by length.

In embodiments, a hint point may be used to assist calculation of a margin line. The hint point may be a point selected by a user. For example, a user may drag a dot or other feature over a tooth of interest (e.g., a preparation tooth under consideration). In one embodiment, the area under interest can be localized using a sequence of operations. Processing logic may perform morphological erosion of a teeth surface (e.g., regions of the 3D surface classified as hard tissue or teeth). This may cause teeth that are adjacent teeth to a tooth associated with a hint point (e.g., over which the hint point has been dragged) would become disconnected by the operation. Processing logic may then take the tooth closest to the hint point and perform morphological dilatation to restore the tooth surface of the tooth proximate to the hint point. If after this process there are still two or more adjacent teeth instead of one tooth that are under consideration, processing logic can cut out the part of the surface that does not fit into a specific bounding box that is determined by the typical size of an average tooth. Subsequently, processing logic may take only those vertices of the found tooth surface that have a high probability of being a margin line (according to the neural network prediction). Now for every connected component of the surface obtained, processing logic can find the corresponding margin line. In embodiments, every such component is either a stripe or a ring around the preparation tooth. In the case of a ring, the ring can be cut and further considered as a stripe or a series of stripes. Processing logic may find the two ends of each stripe, for example by running a wave from its internal point and taking the farthest two points. The final margin line would be a path between the two ends of one or more stripe.

In one embodiment, a path finding operation or algorithm is applied to the 3D model or 3D surface using values from the matrix as a cost basis. Any pathfinding algorithm may be used. Some examples of possible path finding algorithms to use include dynamic programming, Dijkstra's algorithm, A* search algorithm, an incremental heuristic search algorithm, and so on. A pathfinding algorithm may apply a cost function to determine a path of the margin line.

A pathfinding algorithm that uses probability of representing the margin line in the matrix as a cost basis may search for a path with a maximal cost or a path with a minimal cost. The cost function described above searches for minimum cost using a function that is based on an inverse of probability. Alternatively, a cost function may be used that is based directly on probability, where the maximum cost is searched for. If a pathfinding algorithm is run to maximize cost, then a path between vertexes will be determined that results in a maximum aggregate of probability values. The probability scores of the vertexes may be input into the pathfinding algorithm to find the path that has the maximal cost for the probability score. The path finding algorithm may be used to define a contour that represents the margin line. In some embodiments, the margin line may be adjusted to stick to those areas having a maximal curvature.

Other techniques may also be used to compute the margin line based on the assigned probability values and/or confidence values.

At block 815, processing logic grades or scores the probable margin line. In embodiments, processing logic determines grades or scores for each surface point identified as a margin line based on at least one of determined confidence values and/or the cost function. In one embodiment, processing logic determines groups surface points identified as margin line that are proximate with one another and have similar grades/scores into margin line segments (also referred to as margin line portions). Each margin line segment may be assigned a score/grade based on the scores/grades of its constituent surface points.

In one embodiment, at block 820 for each surface point identified as a margin line and/or for each margin line segment, processing logic determines a) a curvature sharpness, b) whether the portion of the margin line comprises an s-shaped curve, c) whether the portion of the margin line is at or proximate to a void in the margin line, and/or d) a number of intraoral scans that are associated with the portion of the margin line. At block 825, processing logic determines one or more of the following for each surface point identified as a margin line and/or for each margin line segment:
a) whether curvature sharpness is below curvature sharpness threshold;
b) whether the surface point or margin line segment comprises an s-shaped curve;
c) whether the surface point or margin line segment is at or proximate to a void in the margin line (e.g., whether the surface point has a distance from a void that is below a distance threshold); and
d) whether the number of intraoral scans associated with surface point or margin line segment is below a threshold number of intraoral scans (e.g., whether the number of intraoral scans used to determine a surface point on the 3D surface is below a threshold).

At block 830, processing logic determines whether the answer to any of a, b, c or d above is yes. If so, then the method continues to block 835 and processing logic reduces the grade/score for those locations for which the answer to a, b, c and/or d was yes. If the answer to a, b, c and d was all no, the method proceeds to block 840.

At block 840, processing logic marks the margin line on the 3D surface. Each margin line segment may be marked using a visualization that is based on the score/grade associated with that margin line segment. In embodiments, margin line segments are divided into three separate score ranges, each of which has an assigned visualization. For example, a low score range may be marked in red, a medium score range may be marked in yellow, and a high score range may be marked in green in embodiments. In some embodiments, for better visual perception, segments that have a size that is below a size threshold can be absorbed into neighbor segments. In some embodiments, processing logic stores the history of segment changes to perform a temporal-based processing on the margin line. This may include averaging of points, a non-decreasing confidence metric, and so on.

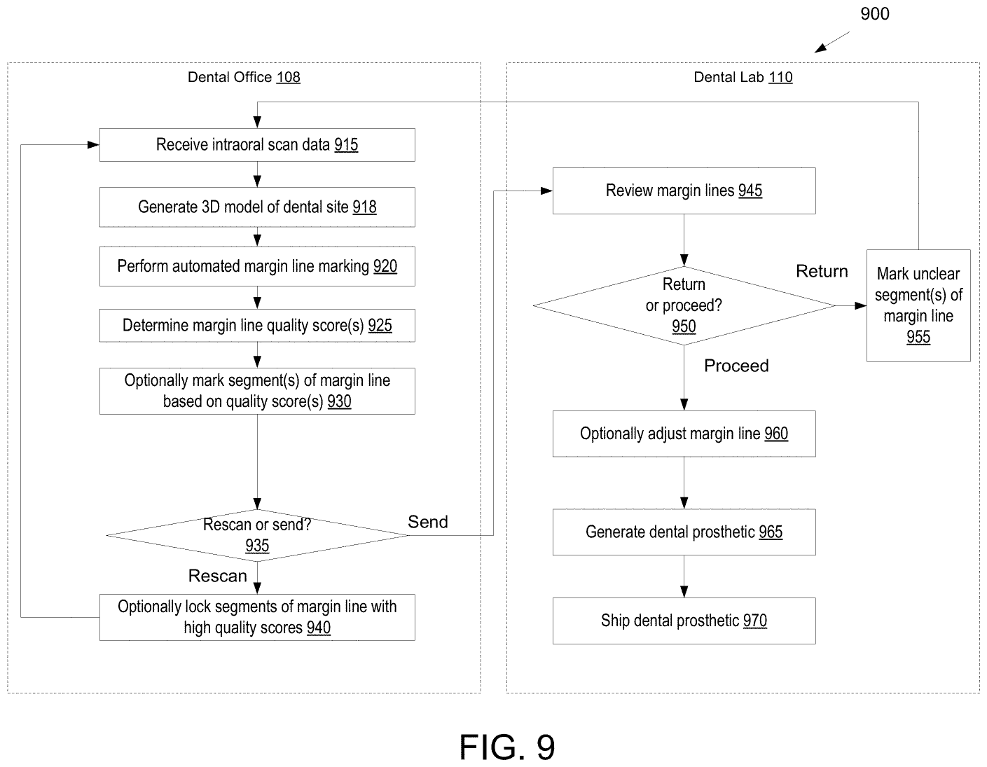

FIG. 9 illustrates an example workflow of a method 900 for generating an accurate virtual 3D model of a dental site and manufacturing a dental prosthetic from the virtual 3D model, in accordance with embodiments of the present disclosure. Operations of the workflow may be performed at a dental office 105 or at a dental lab 110. Those operations performed at the dental office 105 may be performed during a single patient visit or over the course of multiple patient visits. The operations listed under dental office 105 may be performed, for example, by intraoral scan application 115. The operations listed under dental lab 110 may be performed, for example, by dental modeling application 120.

Method 900 may begin at block 915, at which processing logic executing on a computing device associated with dental office 105 receives intraoral scan data. The intraoral scan data may have been generated by intraoral scanner 150 during an intraoral scan process. The intraoral scan data may have been generated in accordance with a standard preparation scanning procedure or in accordance with a partial retraction scanning procedure, as described above. At block 918, processing logic generates a virtual 3D model and/or 3D surface of one or more dental site based on the intraoral scan data, as discussed herein above. The virtual 3D model and/or 3D surface may be of an entire dental arch or of a portion of a dental arch (e.g., a portion including a preparation tooth and adjoining teeth).

At block 920, processing logic performs automated margin line marking on the 3D model and/or 3D surface. In one embodiment, automated margin line marking is performed by first generating appropriate data inputs from the 3D model and/or 3D surface (e.g., one or more images or height maps of the 3D model). These inputs include any information produced during scanning that is useful for margin line detection. Inputs may include image data, such as 2D height maps that provide depth values at each pixel location, and/or color images that are actual or estimated colors for a given 2D model projection. 3D inputs may also be used and include Cartesian location and connectivity between vertices (i.e. mesh). Each image may be a 2D or 3D image generated by projecting a portion of the 3D model or 3D surface that represents a particular tooth onto a 2D surface. Different images may be generated by projecting the 3D model or 3D surface onto different 2D surfaces. In one embodiment, one or more generated images may include a height map that provides a depth value for each pixel of the image. Alternatively, or additionally, intraoral images that were used to generate the 3D model or 3D surface may be used. The generated images and/or the received intraoral scans may be processed by a machine learning model that has been trained to identify margin lines on preparation teeth. The machine learning model may output a probability map that indicates, for each pixel of the image or surface point of the 3D data input into the machine learning model, a probability that the pixel or surface point represents a margin line. In the case of images, the probability map may then be projected back onto the 3D model or 3D surface to assign probability values to points on the 3D model or 3D surface. A cost function may then be applied to find the margin line using the probability values assigned to the points on the 3D model or 3D surface. Other techniques may also be used to compute the margin line based on the assigned probability values.

At block 925, processing logic computes one or more margin line quality scores (also referred to as grades). Each margin line quality score may be based on the cost value for the margin line (or a segment of the margin line) as computed using the cost function. In one embodiment, a margin line quality score is determined for the entirety of the margin line. In one embodiment, multiple additional margin line quality scores are computed, where each margin line quality score is for a particular segment of the margin line. Margin line quality scores may be adjusted according to one or more adjustment criteria, as set forth with reference to FIG. 8.

At block 930, processing logic may mark segments of the margin line on the 3D model or 3D surface based on quality scores. For example, the margin line quality scores for one or more margin line segments may be compared to one or more quality threshold. Any scores that are representative of costs that exceed a maximum cost may fail to satisfy an upper quality threshold. Those segments that fail to satisfy the upper quality threshold may be marked with a marking that distinguishes them from a remainder of the margin line. For example, low quality margin line segments may be highlighted on the 3D model or 3D surface using a red color or other visualization. Other segments having other quality ratings may be represented using other colors and/or visualizations.

At block 935, a doctor may provide feedback indicating that a 3D model is acceptable or that the 3D model should be updated. If the doctor indicates that the 3D model is acceptable, then the 3D model is sent to the dental lab 110 for review, and the method continues to block 945. If the doctor indicates that the 3D model is not acceptable, then the method continues to block 940.

At block 940, the doctor may use a user interface to indicate one or more regions of the 3D model that are to be rescanned. For example, the user interface may include an eraser function that enables the doctor to draw or circle a portion of the 3D model. An area inside of the drawn region or circle may be erased, and a remainder of the 3D model may be locked. Locked regions of the 3D model may not be modified by new intraoral scan data. Alternatively, a one-way lock may be applied, and the locked regions may be modified under certain conditions. Alternatively, processing logic may automatically select regions depicting margin line segments with low quality scores for erasure, and may automatically lock a remainder of the 3D model. Processing logic may then graphically indicate to the doctor where to position the intraoral scanner 150 to generate replacement image data. The method may then return to block 915, and new intraoral image data depicting the region that was erased may be received. The new intraoral image data may be generated using a standard scanning procedure or a partial retraction scanning procedure.

At block 918, the 3D model may be updated based on the new image data. In one embodiment, the unlocked portion of the 3D model is updated based on the new image data, but the locked regions are not updated. In one embodiment, one or more regions are locked using a one-way lock. For the one-way lock, the locked areas are not affected by the new image data if adjusting the regions using the new image data would result in a degraded representation of those regions. However, if use of the new image data would improve the representation of those regions, then the new image data may be used to update those regions. In one embodiment, processing logic processes the new image data (e.g., using a trained machine learning model) to determine a quality score for the new image data. In some embodiments, multiple quality scores are determined for the new image data, where each quality score may be associated with a different region of a dental site. Additionally, quality scores may be determined for one or more regions of the 3D model. If a score for a region from the new image data is higher than the score for that same region from the 3D model, then the image data may be used to update that region of the 3D model. If the score for a region of the new image data is less than or equal to the score for that same region from the 3D model, then the new image data may not be used to update that region of the 3D model.

The operations of blocks 920-935 may then be repeated based on the updated 3D model.

At block 945, a lab technician may review the margin lines in the 3D model (e.g., using a dental modeling application 120). Alternatively, or additionally, processing logic (e.g., processing logic of a dental modeling application 120) may process the 3D model to automatically determine and/or grade the margin line. In one embodiment, reviewing the margin lines at block 945 includes performing operations 920-930. At block 950, processing logic determines whether to proceed with using the 3D model to manufacture a dental prosthetic or to return the 3D model to the dental office 105. If the margin line meets a minimum quality threshold, then the method proceeds to block 960. If the margin line does not meet the minimum quality threshold, then the method continues to block 955, and the 3D model is returned to the dental office 105 to enable the doctor to generate further intraoral scans of the dental site. At block 955, a lab technician may manually mark unclear segments of the margin line. Alternatively, unclear segments may be automatically marked by processing logic at block 955, or may have already been marked at block 945. A message is then sent to the doctor asking for additional intraoral images to be generated. The message may provide a copy of the 3D model showing regions that should be reimaged.

At block 960, the margin line may automatically be adjusted. In some instances, at block 950 processing logic may determine that the margin line has insufficient quality, but for some reason the doctor may be unable to collect new images of the dental site. In such instances, processing logic may proceed to block 960 even if the margin line has an unacceptable level of quality. In such instances, the margin line may be automatically adjusted at block 960. Alternatively, the margin line may be manually adjusted using, for example, CAD tools. In one embodiment, the margin line is adjusted by generating images of the 3D model (e.g., by projecting the 3D model onto 2D surfaces) and processing the images using a trained machine learning model that has been trained to correct margin lines in images of preparation teeth. In one embodiment, one or more operations of method 2000 of FIG. 20 are performed at block 960.

At block 965, processing logic generates a dental prosthetic using the virtual 3D model of the dental site. In one embodiment, the virtual 3D model is input into a rapid prototyping machine (e.g., a 3D printer), and a physical model of the dental site(s) (e.g., of a preparation tooth and adjacent teeth) is produced. The physical 3D model may then be used to generate the dental prosthetic. Alternatively, a virtual 3D model of the dental prosthetic may be generated from the virtual 3D model of the dental site(s), and the virtual 3D model of the dental prosthetic may be used to directly manufacture the dental prosthetic using 3D printing. At block 970, the dental prosthetic may then be shipped to the dental office 105.

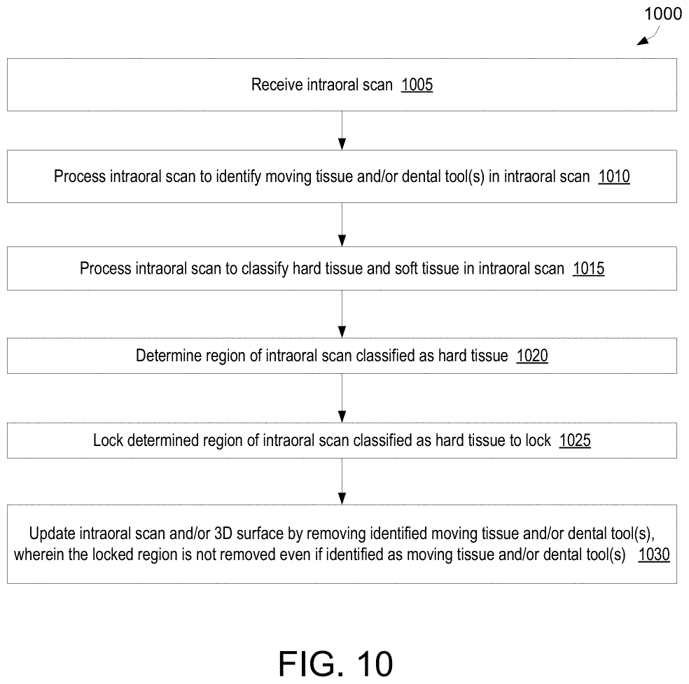

FIG. 10 illustrates a flow diagram for a method 1000 of segmenting intraoral scan data and/or a 3D surface into various classes and updating a/the 3D surface based on the classes, in accordance with an embodiment. At block 1005 of method 1000, processing logic receives an intraoral scan. At block 1010, processing logic processes the intraoral scan to identify moving tissue and/or dental tools in the intraoral scan. In one embodiment, the intraoral scan (or data from the intraoral scan) is input into a trained machine learning model. The machine learning model may then output a probability map that includes, for each point or pixel in the intraoral scan, a probability of the point or pixel being moving tissue and/or a dental tool. In some embodiments, the intraoral scan is registered to a 3D surface, and the 3D surface is updated based on the intraoral scan.

At block 1015, processing logic processes the intraoral scan data and/or the 3D surface to classify hard tissue and soft tissue in the intraoral scan and/or 3D surface. In one embodiment, the intraoral scan (or data from the intraoral scan) or data from the 3D surface is input into a trained machine learning model. The machine learning model may then output a probability map that includes, for each point or pixel in the intraoral scan and/or 3D surface, a first probability of the point belonging to a first class associated with hard tissue and a second probability of the point belonging to a second class associated with soft tissue. In embodiments, a single trained machine learning model performs the operations of both blocks 1010 and 1015.

At block 1020, processing logic determines a region of the intraoral scan and/or 3D surface that was classified as hard tissue (e.g., points or pixels that have a probability of representing hard tissue that is above a threshold, such as 70%, 80%, 90%, etc.). Processing logic may determine a group of adjoining points or pixels classified as hard tissue into a region. Processing logic may then determine an offset inward into the region to determine an updated region of hard tissue.

At block 1025, processing logic locks the determined updated region of the intraoral scan and/or 3D surface that is classified as hard tissue. Locked points will not be removed by algorithms such as moving tissue detection and removal algorithms and/or excess material detection and removal algorithms. At block 1030, processing logic updates the intraoral scan and/or 3D surface by removing the identified moving tissue and/or dental tools from the intraoral scan and/or 3D surface. However, locked region is not removed even if it is also classified as moving tissue or a dental tool.

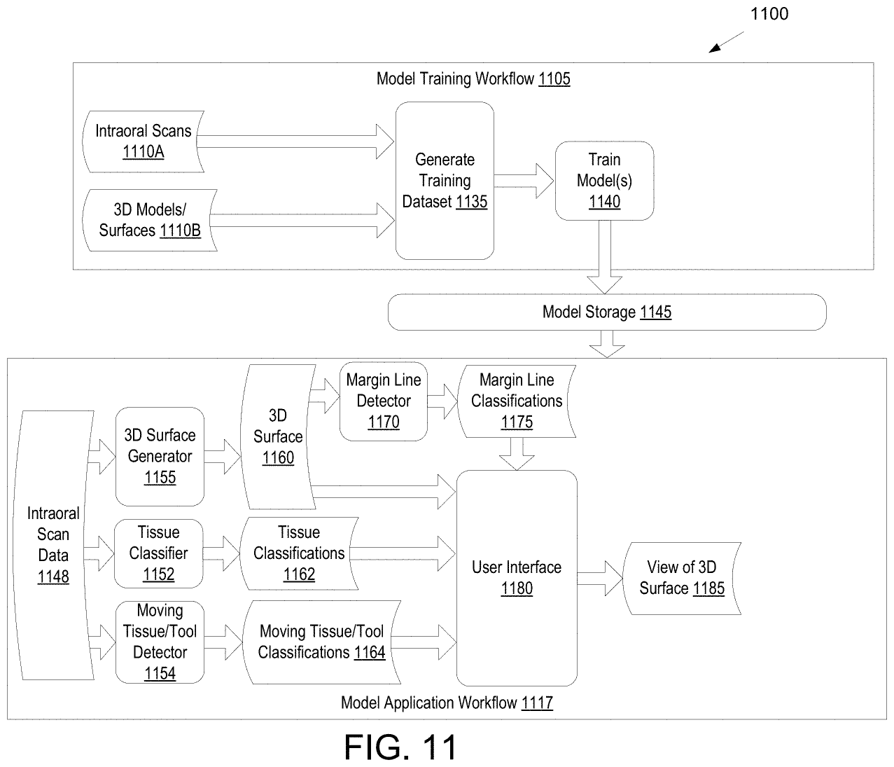

FIG. 11 illustrates a model training workflow 1105 and a model application workflow 1117 for an intraoral scanning application, in accordance with an embodiment of the present disclosure. In embodiments, the model training workflow 1105 may be performed at a server which may or may not include an intraoral scan application, and the trained models are provided to an intraoral scan application (e.g., on computing device 105 of FIG. 1), which may perform the model application workflow 1117. The model training workflow 1105 and the model application workflow 1117 may be performed by processing logic executed by a processor of a computing device. One or more of these workflows 1105, 1117 may be implemented, for example, by one or more machine learning modules implemented in an intraoral scan application 115 or other software and/or firmware executing on a processing device of computing device 1300 shown in FIG. 13.

The model training workflow 1105 is to train one or more machine learning models (e.g., deep learning models) to perform one or more classifying, segmenting, detection, recognition, etc. tasks for intraoral scan data (e.g., 3D scans, height maps, 2D color images, NIRI images, etc.) and/or 3D surfaces generated based on intraoral scan data. The model application workflow 1117 is to apply the one or more trained machine learning models to perform the classifying, segmenting, detection, recognition, etc. tasks for intraoral scan data (e.g., 3D scans, height maps, 2D color images, NIRI images, etc.) and/or 3D surfaces generated based on intraoral scan data. One or more of the machine learning models may receive and process 3D data (e.g., 3D point clouds, 3D surfaces, portions of 3D models, etc.). One or more of the machine learning models may receive and process 2D data (e.g., 2D images, height maps, projections of 3D surfaces onto planes, etc.).

Multiple different machine learning outputs are described herein. Particular numbers and arrangements of machine learning models are described and shown. However, it should be understood that the number and type of machine learning models that are used and the arrangement of such machine learning models can be modified to achieve the same or similar end results. Accordingly, the arrangements of machine learning models that are described and shown are merely examples and should not be construed as limiting.

In embodiments, one or more machine learning models are trained to perform one or more of the below tasks. Each task may be performed by a separate machine learning model. Alternatively, a single machine learning model may perform each of the tasks or a subset of the tasks. Additionally, or alternatively, different machine learning models may be trained to perform different combinations of the tasks. In an example, one or a few machine learning models may be trained, where the trained ML model is a single shared neural network that has multiple shared layers and multiple higher level distinct output layers, where each of the output layers outputs a different prediction, classification, identification, etc. The tasks that the one or more trained machine learning models may be trained to perform are as follows:
I) Dental tissue segmentation—this can include performing point-level classification (e.g., pixel-level classification, voxel-level classification and/or surface point-level classification) of different types of dental tissue from intraoral scans, sets of intraoral scans, 3D surfaces generated from multiple intraoral scans, 3D models generated from multiple intraoral scans, etc. The different types of dental tissue may include, for example, hard tissue (e.g., teeth) and soft tissue (e.g., gingiva or gums).
II) Moving tissue segmentation—this can include performing point-level classification (e.g., pixel-level classification, voxel-level classification and/or surface point-level classification) of moving tissue, dental tools and/or non-moving tissue from sets of intraoral scans, 3D surfaces generated from multiple intraoral scans, 3D models generated from multiple intraoral scans, etc.
III) Margin line identification/marking—this can include performing pixel-level or point-level identification/classification of a margin line around a preparation tooth based on intraoral scans, sets of intraoral scans, 3D surfaces generated from multiple intraoral scans, 3D models generated from multiple intraoral scans, and so on. This can also include marking the identified margin line. Margin line identification and marking is described in US Publication No. 2021/0059796, published Mar. 4, 2021, which is incorporated by reference herein.
IV) Excess material segmentation—this can include performing point-level classification (e.g., pixel-level classification, voxel-level classification and/or surface point-level classification) of excess material from sets of intraoral scans, 3D surfaces generated from multiple intraoral scans, 3D models generated from multiple intraoral scans, etc.

Note that for any of the above identified tasks associated with intraoral scans/3D surfaces/3D models, though they are described as being performed based on an input of intraoral scans, 3D surface and/or 3D models, it should be understood that these tasks may also be performed based on 2D images such as color images, NIRI images, and so on. Any of these tasks may be performed using ML models with multiple input layers or channels, where a first layer may include an intraoral scan/3D surface (or projection of a 3D surface)/3D model (or projection of a 3D model), a second layer may include a 2D color image, a third layer may include a 2D NIRI image, and so on. In another example, a first layer or channel may include a first 3D scan, a second layer or channel may include a second 3D scan, and so on.

One type of machine learning model that may be used to perform some or all of the above asks is an artificial neural network, such as a deep neural network. Artificial neural networks generally include a feature representation component with a classifier or regression layers that map features to a desired output space. A convolutional neural network (CNN), for example, hosts multiple layers of convolutional filters. Pooling is performed, and non-linearities may be addressed, at lower layers, on top of which a multi-layer perceptron is commonly appended, mapping top layer features extracted by the convolutional layers to decisions (e.g. classification outputs). Deep learning is a class of machine learning algorithms that use a cascade of multiple layers of nonlinear processing units for feature extraction and transformation. Each successive layer uses the output from the previous layer as input. Deep neural networks may learn in a supervised (e.g., classification) and/or unsupervised (e.g., pattern analysis) manner. Deep neural networks include a hierarchy of layers, where the different layers learn different levels of representations that correspond to different levels of abstraction. In deep learning, each level learns to transform its input data into a slightly more abstract and composite representation. In an image recognition application, for example, the raw input may be a matrix of pixels; the first representational layer may abstract the pixels and encode edges; the second layer may compose and encode arrangements of edges; the third layer may encode higher level shapes (e.g., teeth, lips, gums, etc.); and the fourth layer may recognize a scanning role. Notably, a deep learning process can learn which features to optimally place in which level on its own. The “deep” in “deep learning” refers to the number of layers through which the data is transformed. More precisely, deep learning systems have a substantial credit assignment path (CAP) depth. The CAP is the chain of transformations from input to output. CAPs describe potentially causal connections between input and output. For a feedforward neural network, the depth of the CAPs may be that of the network and may be the number of hidden layers plus one. For recurrent neural networks, in which a signal may propagate through a layer more than once, the CAP depth is potentially unlimited.

Training of a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many labeled inputs in the training dataset yields a network that can produce correct output when presented with inputs that are different than the ones present in the training dataset. In high-dimensional settings, such as large images, this generalization is achieved when a sufficiently large and diverse training dataset is made available.

For the model training workflow 1105, a training dataset containing hundreds, thousands, tens of thousands, hundreds of thousands or more intraoral scans 1110A, images and/or 3D models/surfaces 11108 should be used to form a training dataset at block 1135. In embodiments, up to millions of cases of patient dentition that may have underwent a prosthodontic procedure and/or an orthodontic procedure may be available for forming a training dataset, where each case may include various labels of one or more types of useful information. Each case may include, for example, data showing a 3D model, intraoral scans, height maps, color images, NIRI images, etc. of one or more dental sites, data showing pixel-level segmentation of the data (e.g., 3D model, intraoral scans, height maps, color images, NIRI images, etc.) into various dental classes (e.g., tooth, restorative object, gingiva, moving tissue, upper palate, etc.), data showing one or more assigned classifications for the data (e.g., scanning role, in mouth, not in mouth, lingual view, buccal view, occlusal view, anterior view, left side view, right side view, etc.), and so on. This data may be processed to generate one or multiple training datasets at block 1135 for training of one or more machine learning models at block 1140.

In one embodiment, generating one or more training datasets 1136 includes gathering one or more intraoral scans with labels 1110A and/or one or more 3D models/surfaces 11108 with labels. The labels that are used may depend on what a particular machine learning model will be trained to do. For example, to train a machine learning model to perform classification of dental sites (e.g., tissue classifier 1152), a training dataset may include pixel-level or point-level labels of hard tissue and soft tissue of dental sites.

Processing logic may gather a training dataset comprising 2D or 3D images, intraoral scans, 3D surfaces, 3D models, height maps, etc. of dental sites (e.g., of dental arches) having one or more associated labels (e.g., pixel-level labeled dental classes in the form of maps (e.g., probability maps), image level labels of dental tissue, etc.). One or more images, scans, surfaces, and/or models and optionally associated probability maps in the training dataset may be resized in embodiments. For example, a machine learning model may be usable for images having certain pixel size ranges, and one or more image may be resized if they fall outside of those pixel size ranges. The images may be resized, for example, using methods such as nearest-neighbor interpolation or box sampling. The training dataset may additionally or alternatively be augmented. Training of large-scale neural networks generally uses tens of thousands of images, which are not easy to acquire in many real-world applications. Data augmentation can be used to artificially increase the effective sample size. Common techniques include random rotation, shifts, shear, flips and so on to existing images to increase the sample size.

To effectuate training, processing logic inputs the training dataset(s) into one or more untrained machine learning models. Prior to inputting a first input into a machine learning model, the machine learning model may be initialized. Processing logic trains the untrained machine learning model(s) based on the training dataset(s) to generate one or more trained machine learning models that perform various operations as set forth above.

Training may be performed by inputting one or more of the images, scans or 3D surfaces (or data from the images, scans or 3D surfaces) into the machine learning model one at a time. Each input may include data from an image, intraoral scan or 3D surface in a training data item from the training dataset. The training data item may include, for example, a height map and an associated probability map, which may be input into the machine learning model. As discussed above, training data items may also include color images, images generated under specific lighting conditions (e.g., UV or IR radiation), and so on. Additionally, pixels of images may include height values or may include both height values and intensity values. The data that is input into the machine learning model may include a single layer (e.g., just height values from a single image) or multiple layers. If multiple layers are used, then one layer may include the height values from the image/scan/surface, and a second layer may include intensity values from the image/scan/surface. Additionally, or alternatively, additional layers may include three layers for color values (e.g., a separate layer for each color channel, such as an R layer, a G layer and a B layer), a layer for pixel information from an image generated under specific lighting conditions, and so on. In some embodiments, data from multiple images/scans/surfaces is input into the machine learning model together, where the multiple images/scans/surfaces may all be of the same dental site. For example, a first layer may include height values from a first scan of a dental site, a second layer may include height values from a second scan of the dental site, a third layer may include height values from a scan of the dental site, and so on.

The machine learning model processes the input to generate an output. An artificial neural network includes an input layer that consists of values in a data point (e.g., intensity values and/or height values of pixels in a height map). The next layer is called a hidden layer, and nodes at the hidden layer each receive one or more of the input values. Each node contains parameters (e.g., weights) to apply to the input values. Each node therefore essentially inputs the input values into a multivariate function (e.g., a non-linear mathematical transformation) to produce an output value. A next layer may be another hidden layer or an output layer. In either case, the nodes at the next layer receive the output values from the nodes at the previous layer, and each node applies weights to those values and then generates its own output value. This may be performed at each layer. A final layer is the output layer, where there is one node for each class, prediction and/or output that the machine learning model can produce. For example, for an artificial neural network being trained to perform dental tissue classification, there may be a first class (hard tissue), and a second class (soft tissue). Other possible classes may include a third class (moving tissue), a fourth class (margin line) and/or one or more additional dental classes (e.g., a fifth class for excess material). Moreover, the class, prediction, etc. may be determined for each pixel or point in the image/scan/surface, may be determined for an entire image/scan/surface, or may be determined for each region or group of pixels of the image/scan/surface. For pixel-level or point-level segmentation, for each pixel/point in the image/scan/surface, the final layer applies a probability that the pixel/point of the image/scan/surface belongs to the first class, a probability that the pixel/point belongs to the second class, and so on.

Accordingly, the output may include one or more prediction and/or one or more a probability map. For example, an output probability map may comprise, for each pixel/point in an input image/scan/surface, a first probability that the pixel belongs to a first dental class, a second probability that the pixel belongs to a second dental class, and so on. For example, the probability map may include probabilities of pixels belonging to tissue classes representing a tooth or a tissue class representing gingiva.

Processing logic may then compare the generated probability map and/or other output to the known probability map and/or label that was included in the training data item. Processing logic determines an error (i.e., a classification error) based on the differences between the output probability map and/or label(s) and the provided probability map and/or label(s). Processing logic adjusts weights of one or more nodes in the machine learning model based on the error. An error term or delta may be determined for each node in the artificial neural network. Based on this error, the artificial neural network adjusts one or more of its parameters for one or more of its nodes (the weights for one or more inputs of a node). Parameters may be updated in a back propagation manner, such that nodes at a highest layer are updated first, followed by nodes at a next layer, and so on. An artificial neural network contains multiple layers of “neurons”, where each layer receives as input values from neurons at a previous layer. The parameters for each neuron include weights associated with the values that are received from each of the neurons at a previous layer. Accordingly, adjusting the parameters may include adjusting the weights assigned to each of the inputs for one or more neurons at one or more layers in the artificial neural network.

Once the model parameters have been optimized, model validation may be performed to determine whether the model has improved and to determine a current accuracy of the deep learning model. After one or more rounds of training, processing logic may determine whether a stopping criterion has been met. A stopping criterion may be a target level of accuracy, a target number of processed images from the training dataset, a target amount of change to parameters over one or more previous data points, a combination thereof and/or other criteria. In one embodiment, the stopping criteria is met when at least a minimum number of data points have been processed and at least a threshold accuracy is achieved. The threshold accuracy may be, for example, 70%, 80% or 90% accuracy. In one embodiment, the stopping criteria is met if accuracy of the machine learning model has stopped improving. If the stopping criterion has not been met, further training is performed. If the stopping criterion has been met, training may be complete. Once the machine learning model is trained, a reserved portion of the training dataset may be used to test the model.

As an example, in one embodiment, a machine learning model (e.g., tissue classifier 1152) is trained to segment intraoral images by classifying regions of those intraoral images into two or more classes. A similar process may be performed to train machine learning models to perform other tasks such as those set forth above. A set of many (e.g., thousands to millions) 3D models and/or intraoral scans of dental arches with labeled dental classes may be collected. In an example, each point in 3D models may include a label having a first value for a first label representing natural teeth, a second value for a second label representing restorative objects, and a third value for a third label representing gums/gingiva. One of the three values may be 1, and the other two values may be 0, for example.

Tissue classifier 1152 may include one or more machine learning models that operate on 3D data or may include one or more machine learning models that operate on 2D data. If tissue classifier 1152 includes a machine learning model that operates on 2D data, then for each 3D model with labeled dental classes, a set of images (e.g., height maps) may be generated. Each image may be generated by projecting the 3D model (or a portion of the 3D model) onto a 2D surface or plane. Different images of a 3D model may be generated by projecting the 3D model onto different 2D surfaces or planes in some embodiments. For example, a first image of a 3D model may be generated by projecting the 3D model onto a 2D surface that is in a top down point of view, a second image may be generated by projecting the 3D model onto a 2D surface that is in a first side point of view (e.g., a buccal point of view), a third image may be generated by projecting the 3D model onto a 2D surface that is in a second side point of view (e.g., a lingual point of view), and so on. Each image may include a height map that includes a depth value associated with each pixel of the image. For each image, a probability map or mask may be generated based on the labeled dental classes in the 3D model and the 2D surface onto which the 3D model was projected. The probability map or mask may have a size that is equal to a pixel size of the generated image. Each point or pixel in the probability map or mask may include a probability value that indicates a probability that the point represents one or more dental classes. For example, there may be two dental classes, including a first dental class representing hard tissue and a second dental class representing soft tissue. Points that have a first dental class may have a value of (1,0) (100% probability of first dental class and 0% probability of second class), and points that have a second dental class may have a value of (0,1). If a machine learning model is being trained to perform image-level classification/prediction as opposed to pixel-level classification/segmentation, then a single value or label may be associated with a generated image as opposed to a map having pixel-level values.

A training dataset may be gathered, where each data item in the training dataset may include an image (e.g., an image comprising a height map) or a 3D surface and an associated probability map (which may be a 2D map if associated with an image or a 3D map if associated with a 3D surface) and/or other label. Additional data may also be included in the training data items. Accuracy of segmentation can be improved by means of additional classes, inputs and multiple views support. Multiple sources of information can be incorporated into model inputs and used jointly for prediction. Multiple dental classes can be predicted concurrently from a single model or using multiple models. Multiple problems can be solved simultaneously: tissue classification, moving tissue detection/classification, margin line detection/classification, excess material classification/detection, etc. Accuracy is higher than traditional image and signal processing approaches.

Additional data may include color image data. For example, for each intraoral scan or image (which may be monochrome), there may also be a corresponding color image. Each data item may include the scan (e.g., a height map) as well as the color image. Two different types of color images may be available. One type of color image is a viewfinder image, and another type of color image is a scan texture. A scan texture may be a combination or blending of multiple different viewfinder images. Each intraoral scan may be associated with a corresponding viewfinder image generated at about the same time that the intraoral image was generated. If blended scans are used, then each scan texture may be based on a combination of viewfinder images that were associated with the raw scans used to produce a particular blended scan.

A default method may be based on depth info only and still allows distinguishing several dental classes such as teeth, gums, moving tissue, excess material, and so on. However, sometimes depth info is not enough for good accuracy. For example, a partially scanned tooth may look like gums or even excess material in monochrome. In such cases color info may help. In one embodiment, color info is used as an additional 3 layers (e.g., RGB), thus, getting 4 layers input for the network. Two types of color info may be used, which may include viewfinder images and scan textures. Viewfinder images are of better quality but need alignment with respect to height maps. Scan textures are aligned with height maps, but may have color artifacts.

Another type of additional data may include an image generated under specific lighting conditions (e.g., an image generated under ultraviolet or infrared lighting conditions). The additional data may be a 2D or 3D image, and may or may not include a height map.

In some embodiments, sets of data points are associated with the same dental site, and are sequentially labeled. In some embodiments a recurrent neural network is used, and the data points are input into a machine learning model during training in ascending order.

In some embodiments, each image or scan includes two values for each pixel in the image, where the first value represents height (e.g., provides a height map), and where the second value represents intensity. Both the height values and the intensity values may be used to train a machine learning model.

In an example, a confocal intraoral scanner may determine the height of a point on a surface (which is captured by a pixel of an intraoral image) based on a focus setting of the intraoral scanner that resulted in a maximum intensity for that point on the surface. The focus setting provides a height or depth value for the point. Typically the intensity value (referred to as a grade) is discarded. However, the intensity value (grade) associated with the height or depth value may be kept, and may be included in the input data provided to the machine learning model.

Once one or more trained ML models are generated, they may be stored in model storage 1145, and may be added to an intraoral scan application (e.g., intraoral scan application 115). Intraoral scan application 115 may then use the one or more trained ML models.

In one embodiment, model application workflow 1117 includes one or more trained machine learning models that function as a tissue classifier 1152, as a moving tissue/tool detector 1154 and/or as a margin line detector 1170. These logics may be implemented as separate machine learning models or as a single combined machine learning model in embodiments. For example, tissue classifier 1152, moving tissue/tool detector 1154 and/or margin line detector 1170 may share one or more layers of a deep neural network. However, each of these logics may include distinct higher level layers of the deep neural network that are trained to generate different types of outputs. The illustrated example is shown with only some of the functionality that is set forth in the list of tasks above for convenience. However, it should be understood that any of the other tasks may also be added to the model application workflow 1117.

For model application workflow 1117, according to one embodiment, an intraoral scanner generates a sequence of intraoral scans 1148. A 3D surface generator 1155 may perform registration between these intraoral scans, stitch the intraoral scans together, and generate a 3D surface 1160 from the intraoral scans. As further intraoral scans are generated, these may be registered and stitched to a 3D surface 1160, increasing a size of the 3D surface 1160 and an amount of data for the 3D surface 1160.

Intraoral scan data 1148 may be input into tissue classifier 1152, which may include a trained neural network. Based on the intraoral scan data 1148, tissue classifier 1152 outputs information on dental site tissue classes 1162, which may be point-level (e.g., pixel-level) classification of the input data. This may include outputting a set of classification probabilities for each pixel/point and/or a single classification for each pixel/point. The output dental site tissue classes 1162 may be, for example, a mask or map of classes and/or of class probabilities. In one embodiment, dental site tissue classifier 1152 identifies for each pixel/point whether it represents hard tissue or soft tissue.

In one embodiment, moving tissue/tool detector receives intraoral scan data 1148 and classifies pixels/points that represent moving tissue (excess tissue). The moving tissue/tool detector may then output moving tissue/tool classifications (e.g., as a probability map or mask).

Margin line detector 1170 may receive data from 3D surface 1160, and may process the data using a trained machine learning model to output a probability map or mask with pixel-level or point-level probabilities of pixels/points belonging to a first class associated with a margin line or a second class not associated with a margin line. Margin line detector 1170 may then grade the classified margin line regions/pixels/points. Accordingly, margin line classifications 1175 may be output by margin line detector 1170.

A user interface 1180 may receive the 3D surface and output a view of the 3D surface 1185. In embodiments, user interface 1180 additionally receives the tissue classifications 1162, the moving tissue/tool classifications 11164 and/or the margin line classifications 1175. User interface 1180 may update the vies of the 3D surface such that a first visualization (e.g., an opaque visualization) is used for hard tissue such as teeth and a second visualization (e.g., a semi-transparent visualization) is used for soft tissue such as gingiva. The user interface 1180 may also remove moving tissue/tools from the 3D surface based on the moving tissue/tool classifications 1164 and may mark margin line segments according to their scores based on margin line classifications 1175 in the view of the 3D surface 1185.

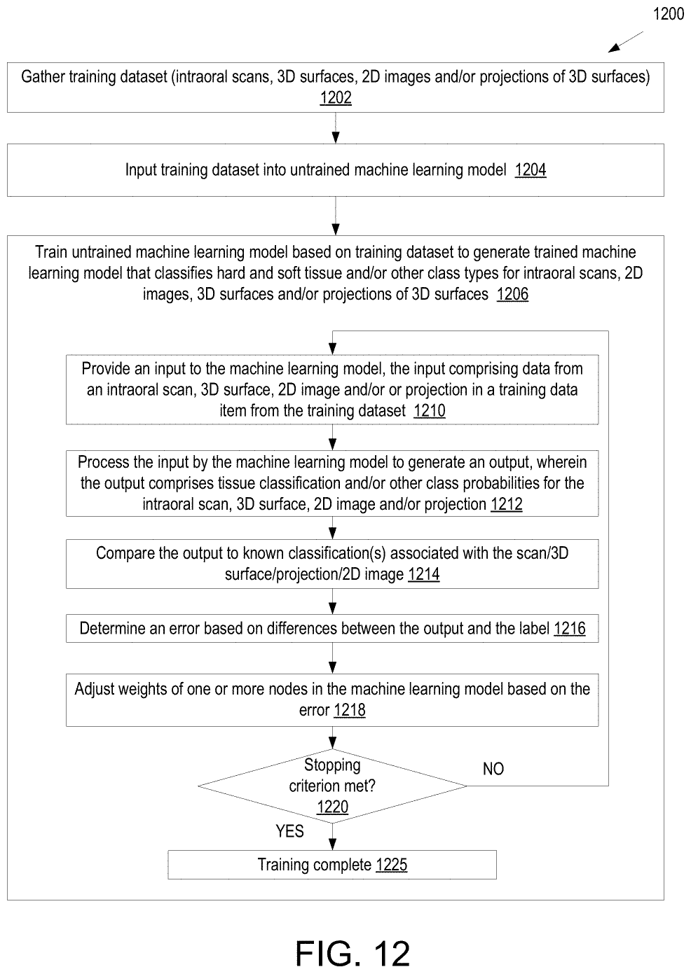

FIG. 12 is a flow chart illustrating an embodiment for a method 1200 of training a machine learning model to identify scanning roles. At block 1202 of method 1200, processing logic gathers a training dataset, which may include intraoral scans (e.g., height maps) of dental sites, 3D surfaces of dental sites, 2D images of dental sites and/or projections of 3D surfaces of dental sites. Each data item (e.g., intraoral scan, image, 3D surfaces, etc.) of the training dataset may include one or more labels. The data items in the training dataset may include pixel-level or point-level labels that indicate one or more dental classes, such as a hard tissue class, a soft tissue class, a moving tissue class, an excess material class and/or a margin line class. Multiple other types of labels may also be associated with the data items in the training dataset.

At block 1204, data items from the training dataset are input into the untrained machine learning model. At block 1206, the machine learning model is trained based on the training dataset to generate a trained machine learning model that classifies scanning roles from intraoral scans, images and/or 3D surfaces (or projections of 3D surfaces). The machine learning model may also be trained to output one or more other types of predictions, image-level classifications, pixel-level classifications, patch-level classifications (where a patch is a group of pixels), decisions, and so on. For example, the machine learning model may also be trained to perform pixel-level classification of intraoral scans, images, 3D surfaces, etc. into dental classes.

In one embodiment, at block 1210 an input of a training data item is input into the machine learning model. The input may include data from an intraoral scan (e.g., a height map), a 3D surface, a 2D image and/or a projection of a 3D surface. At block 1212, the machine learning model processes the input to generate an output. The output may include, for each point or pixel, a first probability that the input belongs to a hard tissue class, a second probability that the input belongs to a soft tissue class, and a third probability that the input belongs to a moving tissue or dental tool class. The output may additionally include, for each pixel or point, a probability of the pixel or point representing a margin line and/or a probability of the pixel or point representing excess material.

At block 1214, processing logic compares, for each point or pixel, the output probabilities to one or more classes associated with the point or pixel in the input. At block 1216, processing logic determines an error based on differences between the output and the label associated with the input. At block 1218, processing logic adjusts weights of one or more nodes in the machine learning model based on the error.

At block 1220, processing logic determines if a stopping criterion is met. If a stopping criterion has not been met, the method returns to block 1210, and another training data item is input into the machine learning model. If a stopping criterion is met, the method proceeds to block 1225, and training of the machine learning model is complete.

FIG. 13 illustrates a diagrammatic representation of a machine in the example form of a computing device 1300 within which a set of instructions, for causing the machine to perform any one or more of the methodologies discussed herein, may be executed. In alternative embodiments, the machine may be connected (e.g., networked) to other machines in a Local Area Network (LAN), an intranet, an extranet, or the Internet. The computing device 1300 may correspond, for example, to computing device 105 and/or computing device 106 of FIG. 1. The machine may operate in the capacity of a server or a client machine in a client-server network environment, or as a peer machine in a peer-to-peer (or distributed) network environment. The machine may be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines (e.g., computers) that individually or jointly execute a set (or multiple sets) of instructions to perform any one or more of the methodologies discussed herein.

The example computing device 1300 includes a processing device 1302, a main memory 1304 (e.g., read-only memory (ROM), flash memory, dynamic random access memory (DRAM) such as synchronous DRAM (SDRAM), etc.), a static memory 1306 (e.g., flash memory, static random access memory (SRAM), etc.), and a secondary memory (e.g., a data storage device 1328), which communicate with each other via a bus 1308.

Processing device 1302 represents one or more general-purpose processors such as a microprocessor, central processing unit, or the like. More particularly, the processing device 1302 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 1302 may also be one or more special-purpose processing devices such as an application specific integrated circuit (ASIC), a field programmable gate array (FPGA), a digital signal processor (DSP), network processor, or the like. Processing device 1302 is configured to execute the processing logic (instructions 1326) for performing operations and steps discussed herein.

The computing device 1300 may further include a network interface device 1322 for communicating with a network 1364. The computing device 1300 also may include a video display unit 1310 (e.g., a liquid crystal display (LCD) or a cathode ray tube (CRT)), an alphanumeric input device 1312 (e.g., a keyboard), a cursor control device 1314 (e.g., a mouse), and a signal generation device 1320 (e.g., a speaker).

The data storage device 1328 may include a machine-readable storage medium (or more specifically a non-transitory computer-readable storage medium) 1324 on which is stored one or more sets of instructions 1326 embodying any one or more of the methodologies or functions described herein, such as instructions for intraoral scan application 1350. A non-transitory storage medium refers to a storage medium other than a carrier wave. The instructions 1326 may also reside, completely or at least partially, within the main memory 1304 and/or within the processing device 1302 during execution thereof by the computer device 1300, the main memory 1304 and the processing device 1302 also constituting computer-readable storage media.

The computer-readable storage medium 1324 may also be used to store intraoral scan application 1350, which may include one or more machine learning modules, and which may perform the operations described herein above. The computer readable storage medium 1324 may also store a software library containing methods for the intraoral scan application 1350. While the computer-readable storage medium 1324 is shown in an example embodiment to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium other than a carrier wave that is capable of storing or encoding a set of instructions for execution by the machine and that cause the machine to perform any one or more of the methodologies of the present disclosure. The term “computer-readable storage medium” shall accordingly be taken to include, but not be limited to, solid-state memories, and optical and magnetic media.

It is to be understood that the above description is intended to be illustrative, and not restrictive. Many other embodiments will be apparent upon reading and understanding the above description. Although embodiments of the present disclosure have been described with reference to specific example embodiments, it will be recognized that the disclosure is not limited to the embodiments described, but can be practiced with modification and alteration within the spirit and scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

1. A system comprising:
an intraoral scanner to generate a plurality of intraoral scans of a dental site during an intraoral scanning session; and
a computing device operatively connected to the intraoral scanner, the computing device to:
receive the plurality of intraoral scans;
generate a three-dimensional (3D) surface of the dental site from the plurality of intraoral scans;
identify hard tissue and soft tissue in at least one of a) the plurality of intraoral scans of the dental site orb) the 3D surface of the dental site; and
display a view of the 3D surface, wherein at least one of a first visualization or a first transparency level is used for first portions of the 3D surface identified as hard tissue and at least one of a second visualization or a second transparency level is used for second portions of the 3D surface identified as soft tissue.

2. The system of claim 1, wherein to identify the hard tissue and the soft tissue the computing device is to:
process at least one of a) the plurality of intraoral scans or b) data from the 3D surface using a trained machine learning model that has been trained to identify hard tissue and soft tissue, wherein the trained machine learning model outputs, for each location in the plurality of intraoral scans or the 3D surface, a first classification indicating hard tissue or a second classification indicating soft tissue.

3. The system of claim 2, wherein the trained machine learning model or a second trained machine learning model is further to output, for each location, a third classification identifying the location as part of a margin line or a fourth classification identifying the location as not being part of the margin line.

4. The system of claim 1, wherein the first visualization comprises an opaque visualization and the second visualization comprises a semi-transparent visualization.

5. The system of claim 4, wherein the hard tissue comprises teeth and the soft tissue comprises gingiva, and wherein scanned portions of the teeth that are below a gum line are visible through the semi-transparent visualization used for the gingiva.

6. The system of claim 1, wherein the dental site comprises a preparation tooth, and wherein the computing device is further to:
identify a margin line around at least a portion of the preparation tooth in at least one of a) one or more of the plurality of intraoral scans or b) data from the 3D surface; and
display the margin line on the 3D surface using one or more additional visualizations.

7. The system of claim 1, wherein the computing device is further to:
receive an additional intraoral scan of the dental site;
add data from the additional intraoral scan to the 3D surface;
update the view of the 3D surface, wherein the data from the additional intraoral scan is semi-transparent in the updated view of the 3D surface;
subsequently segment the data from the additional intraoral scan into hard tissue and soft tissue; and
subsequently update the view of the 3D surface such that the data from the additional intraoral scan associated with hard tissue is opaque and the data from the additional intraoral scan associated with soft tissue remains semi-transparent.

8. The system of claim 1, wherein the computing device is further to:
receive an additional intraoral scan of the dental site;
add data from the additional intraoral scan to the 3D surface;
update the view of the 3D surface, wherein the data from the additional intraoral scan is semi-transparent in the updated view of the 3D surface;
subsequently identify at least one of moving tissue or a dental tool in the data from the additional intraoral scan;
remove at least one of the moving tissue or the dental tool from the 3D surface; and
subsequently update the view of the 3D surface to reflect at least one of the removed moving tissue or the removed dental tool.

9. The system of claim 1, wherein the computing device is further to:
determine whether or not the dental site comprises a preparation tooth;
use a first moving tissue detection algorithm to identify and remove moving tissue from at least one of the plurality of intraoral scans or the 3D surface responsive to determining that a preparation tooth is not detected; and
use a second moving tissue detection algorithm to identify and remove moving tissue from at least one of the plurality of intraoral scans or the 3D surface responsive to determining that a preparation tooth is detected, wherein the second moving tissue detection algorithm is more aggressive at identifying moving tissue than the first moving tissue detection algorithm.

10. The system of claim 1, wherein the second transparency level comprises 100% transparency, and wherein the second portions of the 3D surface identified as soft tissue are not visible due to the 100% transparency.

11. The system of claim 1, wherein the 3D surface is a 3D surface of a preparation tooth, and wherein the computing device is further to:
overlay the 3D surface onto a second 3D surface of a dental arch that includes the preparation tooth, wherein gums from the second 3D surface are shown using a semi-transparent visualization.

12. The system of claim 1, wherein the computing device is further to:
receive a user input of a coordinate;
determine a first tooth closest to the coordinate;
use at least one of the first visualization or the first transparency level for displaying the first tooth closest to the coordinate; and
use at least one of the second visualization or the second transparency level for displaying a second tooth.

13. The system of claim 12, wherein the computing device is further to:
receive a new user input of a new coordinate;
determine that the second tooth is a closest tooth to the new coordinate;
use at least one of the first visualization or the first transparency level for displaying the second tooth closest to the new coordinate; and
use at least one of the second visualization or the second transparency level for displaying the first tooth.

14. The system of claim 12, wherein at least one of a mesial surface or a distal surface of the first tooth is visible through the second tooth displayed using at least one of the second visualization or the second transparency level.

15. The system of claim 12, wherein receiving the user input of the coordinate comprises receiving user input dragging a hint feature to the coordinate.

16. The system of claim 12, wherein the first tooth is a preparation tooth having a margin line, and wherein determining the first tooth closest to the coordinate comprises:
identifying a margin line of the preparation tooth;
determining that a point on the 3D surface closest to the coordinate is within the margin line in a plane; and
classifying points on the preparation tooth that are within the margin line as the preparation tooth.

17. The system of claim 12, wherein determining the first tooth closest to the coordinate comprises:
performing one or more morphological operations to divide the 3D surface into a plurality of parts that correspond to distinct teeth;
finding a point on the 3D surface closest to the coordinate; and
selecting a part associated with the first tooth that comprises the point on the 3D surface closest to the coordinate.

18. A non-transitory computer readable medium comprising instructions that, when executed by a processing device, cause the processing device to perform operations comprising:
receiving a plurality of intraoral scans of a dental site during an intraoral scanning session;
generating a three-dimensional (3D) surface of the dental site from the plurality of intraoral scans;
identifying hard tissue and soft tissue in at least one of a) the plurality of intraoral scans of the dental site orb) the 3D surface of the dental site; and
displaying a view of the 3D surface, wherein at least one of a first visualization or a first transparency level is used for first portions of the 3D surface identified as hard tissue and at least one of a second visualization or a second transparency level is used for second portions of the 3D surface identified as soft tissue.

19. The non-transitory computer readable medium of claim 18, wherein identifying the hard tissue and the soft tissue comprises:
processing at least one of a) the plurality of intraoral scans or b) data from the 3D surface using a trained machine learning model that has been trained to identify hard tissue and soft tissue, wherein the trained machine learning model outputs, for each location in the plurality of intraoral scans or the 3D surface, a first classification indicating hard tissue or a second classification indicating soft tissue.

20. The non-transitory computer readable medium of claim 19, wherein the trained machine learning model or a second trained machine learning model further outputs, for each location, a third classification identifying the location as part of a margin line or a fourth classification identifying the location as not being part of the margin line.

21. The non-transitory computer readable medium of claim 18, wherein the first visualization comprises an opaque visualization and the second visualization comprises a semi-transparent visualization.

22. The non-transitory computer readable medium of claim 21, wherein the hard tissue comprises teeth and the soft tissue comprises gingiva, and wherein scanned portions of the teeth that are below a gum line are visible through the semi-transparent visualization used for the gingiva.

23. The non-transitory computer readable medium of claim 18, wherein the dental site comprises a preparation tooth, the operations further comprising:
identifying a margin line around at least a portion of the preparation tooth in at least one of a) one or more of the plurality of intraoral scans or b) data from the 3D surface; and
displaying the margin line on the 3D surface using one or more additional visualizations.

24. A method comprising:
receiving a plurality of intraoral scans of a dental site during an intraoral scanning session;
generating a three-dimensional (3D) surface of the dental site from the plurality of intraoral scans;
identifying hard tissue and soft tissue in at least one of a) the plurality of intraoral scans of the dental site orb) the 3D surface of the dental site; and
displaying a view of the 3D surface, wherein at least one of a first visualization or a first transparency level is used for first portions of the 3D surface identified as hard tissue and at least one of a second visualization or a second transparency level is used for second portions of the 3D surface identified as soft tissue.

25-74. (canceled)
